(19) United States
(12) Patent Application Publication
(10) Pub. No.: US 2024/0409517 A1
(43) Pub. Date: Dec. 12, 2024

(54) ORGANIC COMPOUNDS

(71) Applicant: GIVAUDAN SA, VERNIER (CH)

(72) Inventors: Stephan HAIBER, Naarden (NL); Esther Maria VREMAN, Naarden (NL); Adri DE KLERK, Naarden (NL); Luis IBANEZ GONZALEZ, Kemptthal (CH); Abdelmajid KAOUAS, Naarden (NL); Eric KOHLEN, Naarden (NL); Jose LHERONDEL, Peymeinade (FR); Lisa Maria WIJNEN, Naarden (NL); Cornelis WINKEL, Naarden (NL)

(21) Appl. No.: 18/699,783

(22) PCT Filed: Oct. 13, 2022
(86) PCT No.: PCT/EP2022/078460

§ 371 Date: Apr. 9, 2024

(30) Foreign Application Priority Data
Oct. 15, 2021 (GB) 2114766.5

(51) Int. Cl. C07D 233/90; A23L 27/00; A23L 27/20; C07D 239/28; C07D 401/12; C07D 403/12; C07D 405/12; C07D 409/12; C07D 417/12

(52) U.S. Cl. CPC C07D 233/90; A23L 27/2054; A23L 27/2056; A23L 27/84; A23L 27/86; A23L 27/88; C07D 239/28; C07D 401/12; C07D 403/12; C07D 405/12; C07D 409/12; C07D 417/12

(57) ABSTRACT

The invention relates to the use of compounds of formula (I) and edible salts thereof, wherein

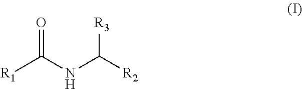

R₁ is selected from the group consisting of 2-(1H-4-imidazolyl)-ethenyl, 1H-5-indolyl, 2-(1H-5-imidazolyl)-ethenyl, 1-amino-2-(1H-4-imidazolyl)-ethyl, (1,3-thiazol-2-yl)-ethenyl, 2,3-dihydro-1H-indol-2-yl, 2-(pyrimidin-2-yl)ethenyl, heptadecanyl, 1-heptadec-8-enyl, heptadeca-8,11-dienyl, heptadeca-8,11,14-trienyl, 2-(4H-imidazol-2-yl)-ethenyl, 2-(4H-imidazol-2-yl)-ethyl, 2-phenyl-ethenyl, 2-(furan-2-yl)-ethenyl, 2-(thiophen-2-yl)-ethenyl, 2-(thiophen-3-yl)-ethenyl, 2-but-2-enoyl, 2-butyl, 2,6-dimethylhepta-1,5-dienyl, 1,3-benzothiazol-6-yl;
R₂ is selected from the group consisting of (1H-imidazol-4-yl)-methyl, (1H-3-indol-3-yl)-methyl, 4-hydroxybenzyl, methylsulfanylethyl, hydroxymethyl, CH₂—COOH, (pyridin-4-yl)methyl, (pyridin-2-yl)methyl, 1-(2,6-dimethylhepta-1,5-dienyl), 2-(pyrimidin-2-yl)methyl, (1H-imidazol-5-yl)-methyl, (1H-imidazol-2-yl)-methyl, (1H-pyrrol-2-yl)-methyl, phenyl, pyrimidin-5-yl, pyrazin-2-yl;
R₃ is selected from the group consisting of H, COOH, or a compound selected from the group consisting of 3-(1H-imidazol-4-yl) prop-2-enamide, methyl 2,3-dihydro-1H-indole-2-carboxylate, and 3,6-bis[(1H-imidazol-4-yl)methyl]piperazine-2,5-dione,
as flavor modulating compound, and to flavor compositions and consumer products comprising said compounds.

### TECHNICAL FIELD

The present invention relates generally to novel compounds and edible salts thereof, which are useful flavor modulating compounds. The invention further relates to flavor compositions and consumer products like foodstuffs or beverages comprising said compounds. The invention also relates to the use of said compounds, and to a method to confer, enhance, improve, complement or modify the flavor properties of a flavor composition or a consumer product by using said compounds.

### BACKGROUND

Saltiness is one of the five basic taste attributes next to umami, sweet, bitter and sour. Salty taste is associated with sodium chloride and is essential to stimulate the uptake of healthy minerals by food. However, modern diets are rich in strongly processed foods which often contain very high amounts of sodium chloride. According to the World Health Organization (WHO) an average daily intake of 5 g/day is recommended. According to the FDA the personal average daily intake (PADI) of sodium chloride for an American is more than 8 g sodium chloride per day, which is clearly above the recommended value. Continuously high PADI of sodium chloride can cause negative side effects like hypertension, cardiovascular disease, kidney failure and stroke. It is highly desirable to reduce the PADI of sodium chloride, preferably without compromising the desired salty taste. Therefore, there is a need for compounds which enhance the salty taste perception.

A range of candidate flavor compounds for salt enhancement have been described in literature. For example, in U.S. Pat. No. 9,155,329B2 low molecular weight amides with additional hydroxyl or alkoxyl groups are disclosed to have salt enhancing properties.

The at least partial replacement of sodium chloride by other salts like potassium chloride is often associated with negative taste aspects (Khetra et al, Int. Dairy J. (2019), 91, 165-171).

Another relevant aspect that is sometimes mixed up with salt perception is umami taste, although completely different receptor mechanisms are involved. The most prominent example for compounds imparting umami taste is monosodium glutamate (MSG) (Ikeda, J. Tokyo Chem. Soc. (1909), 30, 820-836). Umami taste can be modified by the ribonucleotides inosine monophosphate (IMP) and guanosine monophosphate (GMP) (Kodama, J. Chem. Soc. of Japan. (1913), 34:751-757; Kuninaka, J. Agric. Chem. Soc. Jpn. (1960), 34, 487-492). Other compounds which enhance umami taste are for example theanine (Suzuku et al. J Agric Food Chem. (2002), 50 313-318) or a special oligopeptide (Yamasaki, et al, Agric. Biol. Chem. (1978), 42, 1761-1765; Tamura et al, Agric. Biol. Chem. (1989), 53, 319-325).

Glutathione is described to influence umami and saltiness at the same time, more precisely the duration of the taste stimulus (Tazuko et al, Chem. Senses (2016), Volume 41, 623-630). A group of general taste enhancers was reported in EP1291342.

A variety of other molecules is known to improve or to influence salt perception. Also volatile aroma compounds can help to mimic an increased saltiness (Batenburg et al, J. Food Sci. (2011), 76, 280-288).

There remains a need for new taste improving substances to be added to flavorist's palette which can modulate flavor.

### SUMMARY

In accordance with a first aspect of the present invention there is provided the use of a compound of formula (I)

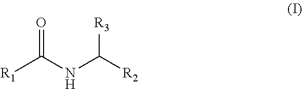

or an edible salt thereof as flavor modulating compound.

In accordance with a second aspect of the present invention there is provided a flavor composition comprising said compound.

In accordance with a third aspect of the present invention there is provided a consumer product comprising said compound or said flavor composition.

In accordance with a fourth aspect of the present invention there are provided novel compounds as flavor modulating compounds.

In accordance with a fifth aspect of the present invention there is provided a method to confer, enhance, improve or modify the flavor properties of a flavor composition or a consumer product.

Certain embodiments of any aspect of the present invention may provide one or more of the following advantages:
modulation, in particular positive modulation, of salty taste perception,
reduction of bitter taste, for example of KCl,
reduction of sour taste, for example of NH₄Cl,
enhanced perception of umami taste, and
enhanced aroma perception of foodstuffs or beverages, in particular of savoury foodstuffs or beverages.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that certain peptides, most of them derived from desaminated and/or decarboxylated amino acids, have flavor modulating properties.

There is therefore provided a use of one or more compounds of formula (I)

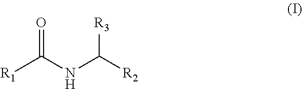

and edible salts thereof, wherein

R₁ is selected from the group consisting of 2-(1H-4-imidazolyl)-ethenyl, 1H-5-indolyl, 2-(1H-5-imidazolyl)-ethenyl, 1-amino-2-(1H-4-imidazolyl)-ethyl, (1,3-thiazol-2-yl)-ethenyl, 2,3-dihydro-1H-indol-2-yl, 2-(pyrimidin-2-yl)ethenyl, heptadecanyl, 1-heptadec-8-enyl, heptadeca-8,11-dienyl, heptadeca-8,11,14-trienyl, 2-(4H-imidazol-2-yl)-ethenyl, 2-(4H-imidazol-2-yl)-ethyl, 2-phenyl-ethenyl, 2-(furan-2-yl)-ethenyl, 2-(thiophen-2-yl)-ethenyl, 2-(thiophen-3-yl)-ethenyl, 2-but-2-enoyl, 2-butyl, 2,6-dimethylhepta-1,5-dienyl, 1,3-benzothiazol-6-yl;

R₂ is selected from the group consisting of (1H-imidazol-4-yl)-methyl, (1H-3-indol-3-yl)-methyl, 4-hydroxybenzyl, methylsulfanylethyl, hydroxymethyl, CH₂-COOH, (pyridin-4-yl)methyl, (pyridin-2-yl)methyl, 1-(2,6-dimethylhepta-1,5-dienyl), 2-(pyrimidin-2-yl)methyl, (1H-imidazol-5-yl)-methyl, (1H-imidazol-2-yl)-methyl, (1H-pyrrol-2-yl)-methyl, phenyl, pyrimidin-5-yl, pyrazin-2-yl,

R₃ is selected from the group consisting of H, COOH,

or a compound selected from the group consisting of 3-(1H-imidazol-4-yl) prop-2-enamide, methyl 2,3-dihydro-1H-indole-2-carboxylate, and 3,6-bis[(1H-imidazol-4-yl)methyl]piperazine-2,5-dione,

as flavor modulating compound.

As used herein, edible salts include those typically employed in the food and beverage industry and include chlorides, sulphates, phosphates, gluconates, sodium, citrates, carbonates, acetates and lactates.

If the compound of the invention contains stereo centers or double bonds, the compound is a single isomer, for example an enantiomer or a diastereomer or double bond isomer, or a mixture of more than one isomer. In particular, the double bond of the compounds of the present invention can have either E or Z-configuration, or the compound is present as a mixture.

If the compound of the present invention can exist in the form of tautomers, both tautomeric forms are encompassed by the present invention. For example, the imidazole moieties of histidine and derivatives thereof exist in two tautomeric forms 1H-imidazol-4-yl and 3H-imidazol-4-yl.

Furthermore, the compound of the invention may be a mixture of more than one chemical compound in the form of any one of its isomers or a mixture thereof.

The compound of formula (I) is an amide or a peptide, characterized by the amide group —C(═O)N═, which is formed by an amino nitrogen atom of an amino acid residue and a carbonyl carbon atom of another amino acid residue. Alternatively, the carbonyl carbon atom is provided from a fatty acid.

For example, the amino acid residue (R₁) connected to the carbon atom of the amide group of the compound of formula (I) is a residue of an amino acid selected from the group consisting of Histidine, and desaminated and dehydrogenated Histidine.

For example, the residue R₁ derived from fatty acids have one or more CC double bonds. Those double bonds can be in E- or Z-configuration, or making a mixture of isomers. For example, the CC double bond configuration of one or more double bonds is Z.

For example, the amino acid residue connected to the nitrogen atom of the amide group of the compound of formula (I) is a residue of an amino acid selected from the group consisting of β-Alanine, Histidine, Tryptophan, Tyrosine and decarboxylated, Histidine, Serine, Methionine and Tyrosine.

The compounds of the present invention described above possess flavor modulating properties. For example, some of the compounds can enhance the salty taste perception, and/or reduce bitter taste and/or enhance umami taste.

Throughout this document the terms “taste” and “flavor” are used interchangeably to describe the sensory impact that is perceived via the mouth, especially the tongue, and the olfactory epithelium in the nasal cavity.

As used herein, the term “flavor modulating compound” refers to a compound that has no flavor properties as such. However, said substance is capable of altering or complementing or modulating the taste impact of other flavoring substances contained in a flavor composition or in a consumer product, including the salty taste impact, acidic taste impact, bitterness and/or umami taste impact.

For example, the flavor modulating compound does not have any salty taste at all up to levels above 1000 ppm. In combination with salty flavor compounds, for example with NaCl, it can enhance the salty taste perception.

For example, the flavor modulating compound does not have any recognizable taste at a level above 1000 ppm for counteracting or masking bitter taste. In combination with compounds which can have bitter off taste, for example KCl, the bitter taste is reduced.

For example, the flavor modulating compound does not have any recognizable taste at a level above 1000 ppm for counteracting or masking sour taste. In combination with compounds which can have sour off taste, for example NH₄Cl, the sour taste is reduced.

For example, the flavor modulating compound does not impart any umami taste at a level above 1000 ppm. In combination with compounds which can impart umami taste, for example MSG, the umami taste is enhanced.

As used herein, the term “flavoring substance” refers to any substance that is capable of imparting a detectable flavor impact, especially at a concentration below 0.1 wt. %, more preferably below 0.01 wt. %. For example, such flavoring substance may be selected from natural flavors, artificial flavors, spices, seasonings, and the like, synthetic flavor oils and flavor aromatics and/or oils, oleoresins, essences, distillates, and extracts derived from plants, leaves, flowers, fruits, and so forth, Generally, any flavor or food additive such as those described in Chemicals Used in Food Processing, publication 1274, pages 63-258, by the National Academy of Sciences, can be used. This publication is incorporated herein by reference.

It was found that the flavor modulating compounds of the invention are very useful ingredients which are capable in the presence of other flavoring substances to impart highly appreciated taste sensations to the products in which they are incorporated, specifically “roundness”, “fullness”, “substance”, “transparency”, “complexity”, “expanding”, “continuity”, “long lasting”, 20) “tingling”, “numbing”, “bitter” and/or “metallic”. Because of this, the present taste improving substances can be employed to improve the taste (including “mouthfeel”) of foodstuffs and beverages.

In a further aspect, there is provided the use of one or more compounds of formula (I) and edible salts thereof as flavor modulating compounds, as defined above, wherein the compound possess at least one aromatic unit attached to a residue via an amide bond. For example, the aromatic unit can be selected from the group consisting of phenyl, imidazole, thiazole, indole, furan, thiophen, benzothiazol, pyrimidine, pyrazine, pyrrol. The aromatic unit can be a moiety of R₁ and/or R₂. In one embodiment, the aromatic unit is imidazole.

In a further aspect, there is provided the use of one or more compounds of formula (I) and edible salts thereof as flavor modulating compounds, as defined above, wherein the compound possess two aromatic units attached to a residue via an amide bond, and wherein the aromatic unit can be a moiety of R₁ and R₂. For example, the aromatic unit can be selected from the group consisting of phenyl, imidazole, thiazole, indole, furan, thiophen, benzothiazol, pyrimidine, pyrazine, pyrrol. In one embodiment, at least one of the two aromatic units attached to a residue via an amide bond is imidazole.

In a further aspect, there is provided the use of one or more compounds of formula (I) and edible salts thereof as flavor modulating compounds, wherein the compounds are represented by formula (II) in the form of any one of its isomers or a mixture thereof

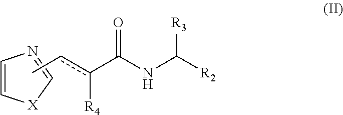

wherein

[symbol] is indicating a carbon-carbon single or double bond,

X is representing a heteroatom selected from the group consisting of N and S,

R₄ is either H or NH₂,

and R₂ and R₃ have the same meaning as defined for the compound of formula (I).

The compounds of formula II possess a five membered unsaturated heterocycle with two heteroatoms attached to a residue via an amide bond. For example, the five membered unsaturated heterocycle is imidazole or thiazole.

In a further aspect, there is provided the use of one or more compounds of formula (I) and edible salts thereof as flavor modulating compounds, wherein the compounds are represented by formula (III) in the form of any one of its isomers or a mixture thereof,

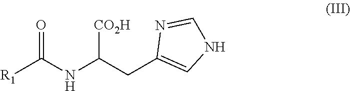

wherein R₁ is selected from the group consisting of heptadecanyl, 1-heptadec-8-enyl, heptadeca-8,11-dienyl and heptadeca-8,11,14-trienyl.

The compounds of formula (III) are derived from Histidine and fatty acids. The fatty acids might be saturated or having one or more double bonds. The compounds of formula (III) are taste modulating compounds, in particular they can enhance salty taste.

In a further embodiment of the present invention, there is provided the use of a compound according to formula (I) as defined above or the additional compounds, wherein the compound is selected from the group consisting of 3-(1H-imidazol-4-yl)-N-[2-(1H-imidazol-4-yl)ethyl]prop-2-enamide, N-[2-(1H-imidazol-4-yl) ethyl]-1H-indole-5-carboxamide, Histidyltyrosine, N-[3-(1H-imidazol-5-yl) prop-2-enoyl]-tryptophan, Histidylhistidine, N-[3-(methylsulfanyl)propyl]histidinamide, N-(2-hydroxyethyl) histidinamide, Histidyl-β-alanine, N-[octadeca-9,12-dienoyl]histidine, N-[2-(1H-imidazol-4-yl)ethyl]-3-(1,3-thiazol-2-yl)prop-2-enamide, N-[octadeca-9,12,15-trienoyl]histidine, N-[octadec-9-enoyl]histidine, N-octadecanoylhistidine, 3-(1H-imidazol-4-yl)prop-2-enamide, 3-(1H-imidazol-4-yl)-N-[2-(pyridin-4-yl)ethyl]prop-2-enamide, methyl 2,3-dihydro-1H-indole-2-carboxylate, 3-(1H-imidazol-4-yl)-N-[2-(pyridin-2-yl)ethyl]prop-2-enamide, N-[3,7-dimethylocta-2,6-dien-1-yl]-3-(1H-imidazol-4-yl)prop-2-enamide, N-[2-(1H-imidazol-4-yl)ethyl]-2,3-dihydro-1H-indole-2-carboxamide, 3-(pyrimidin-2-yl)-N-[2-(pyrimidin-2-yl)ethyl]prop-2-enamide, 3,6-bis[(1H-imidazol-4-yl)methyl]piperazine-2,5-dione, 3-(4H-imidazol-2-yl)-N-[2-(1H-imidazol-5-yl) ethyl]prop-2-enamide, 3-(1H-imidazol-4-yl)-N-[2-(1H-imidazol-2-yl)ethyl]prop-2-enamide, N-(2-hydroxyethyl)-3-(1H-imidazol-4-yl)propanamide, 3-(1H-imidazol-4-yl)-N-[2-(1H-pyrrol-2-yl)ethyl]prop-2-enamide, 3-[2-amino-3-(1H-imidazol-4-yl)propanamido]propanoic acid, N-[2-(1H-imidazol-4-yl) ethyl]-3-phenylprop-2-enamide, N-benzyl-3-(1H-imidazol-4-yl)prop-2-enamide, 3-(1H-imidazol-4-yl)-N-[(pyrimidin-5-yl)methyl]prop-2-enamide, 3-(1H-imidazol-4-yl)-N-[(pyrazin-2-yl)methyl]prop-2-enamide, 3-(furan-2-yl)-N-[2-(1H-imidazol-4-yl)ethyl]prop-2-enamide, N-[2-(1H-imidazol-4-yl)ethyl]-3-(thiophen-2-yl)prop-2-enamide, N-[2-(1H-imidazol-4-yl)ethyl]-3-(thiophen-3-yl)prop-2-enamide, 3-(1H-imidazol-4-yl)-2-{[(2E)-3-phenylprop-2-enoyl]amino}propanoic acid, 3-(1H-imidazol-4-yl)-2-{[(2E)-2-methylbut-2-enoyl]amino}propanoic acid, N-[2-(1H-imidazol-4-yl)ethyl]-2-methylbutanamide, N-[2-(1H-imidazol-4-yl)ethyl]-3,7-dimethylocta-2,6-dienamide, 2-{[3-(furan-2-yl)prop-2-enoyl]amino}-3-(1H-imidazol-4-yl)propanoic acid, 3-(1H-imidazol-5-yl)-2-{[3-(1H-imidazol-4-yl)prop-2-enoyl]amino}propanoic acid, N-[2-(1H-imidazol-5-yl)ethyl]-1,3-benzothiazole-6-carboxamide, 3-(1H-imidazol-4-yl)-N-(2-phenylethyl) prop-2-enamide.

In particular, there is provided the use of a compound according to formula (I) as defined above or the additional compounds, wherein the compound is selected from the group consisting of (2Z)-or (2E)-3-(1H-imidazol-4-yl)-N-[2-(1H-imidazol-4-yl) ethyl]prop-2-enamide, N-[2-(1H-imidazol-4-yl)ethyl]-1H-indole-5-carboxamide, Histidyltyrosine, N-[3-(1H-imidazol-5-yl)prop-2-enoyl]-tryptophan, Histidylhistidine, N-[3-(methylsulfanyl)propyl]histidinamide, N-(2-hydroxyethyl)histidinamide, Histidyl-B-alanine, N-[2-(1H-imidazol-4-yl)ethyl]-3-(1,3-thiazol-2-yl)prop-2-enamide, 3-(1H-imidazol-4-yl)prop-2-enamide, 3-(1H-imidazol-4-yl)-N-[2-(pyridin-4-yl)ethyl]prop-2-enamide, methyl 2,3-dihydro-1H-indole-2-carboxylate, 3-(1H-imidazol-4-yl)-N-[2-(pyridin-2-yl)ethyl]prop-2-enamide, N-[3,7-dimethylocta-2,6-dien-1-yl]-3-(1H-imidazol-4-yl)prop-2-enamide, N-[2-(1H-imidazol-4-yl)ethyl]-2,3-dihydro-1H-indole-2-carboxamide, 3-(pyrimidin-2-yl)-N-[2-(pyrimidin-2-yl)ethyl]prop-2-enamide, 3,6-bis[(1H-imidazol-4-yl)methyl]piperazine-2,5-dione.

In a further embodiment of the present invention, there is provided the use of a mixture of compounds according to formula (I) as defined above as flavor modulating compounds.

In a further embodiment the flavor modulating compounds according to the present invention are particularly useful in a wide variety of flavor compositions and consumer products including savoury food, non-savoury food, such as dairy, beverages and confectionery.

For example, a flavor composition comprises at least 0.01 wt. %, or at least 0.1 wt %, or at least 0.5 wt % of flavoring substances based on the total weight of the composition, and between 0.001 and 80 wt % of the flavor modulating compounds according to the present invention, preferably between 0.01 and 50 wt. %, more preferably between 0.01 and 20 wt. % of the flavor modulating substances based on the total weight of the composition.

In a typical flavor composition, the flavor modulating compounds and flavoring substances are employed in a weight ratio within the range of 10:1 to 1:150, preferably in a weight ratio of 5:1 to 1:100.

The flavor compositions comprising the flavor modulating compound may suitably be prepared in the form of a liquid, a paste or a powder. For example, the flavor composition is a free flowing powder.

Typical examples of flavor compositions include savoury flavorings, sour/acid flavorings and others.

The flavor modulating compounds of the invention can be used in flavor compositions in conjunction with one or more ingredients or excipients conventionally used in flavor compositions beside flavoring substances, for example carrier materials and other auxiliary agents commonly used in the art. Suitable excipients for flavor compositions are well known in the art and include, for example, without limitation, solvents (including water, alcohol, ethanol, oils, fats, vegetable oil, and miglyol), binders, diluents, disintegranting agents, lubricants, flavor agents, coloring agents, preservatives, antioxidants, emulsifiers, stabilisers, flavor-enhancers, anti-caking agents, and the like.

Examples of such carriers or diluents for flavor compositions may be found in for example, “Perfume and Flavour Materials of Natural Origin”, S. Arctander, Ed., Elizabeth, N.J., 1960; in “Perfume and Flavour Chemicals”, S. Arctander, Ed., Vol. I & II, Allured Publishing Corporation, Carol Stream, USA, 1994; in “Flavourings”, E. Ziegler and H. Ziegler (ed.), Wiley-VCH Weinheim, 1998, and “CTFA Cosmetic Ingredient Handbook”, J.M. Nikitakis (ed.), 1st ed., The Cosmetic, Toiletry and Fragrance Association, Inc., Washington, 1988.

Other suitable and desirable ingredients of flavor compositions are described in standard texts, such as “Handbook of Industrial Chemical Additives”, ed. M. and I. Ash, 2nd Ed., (Synapse 2000).

In another aspect of the present invention, there is further provided a consumer product comprising at least one compound of formula (I) or a flavor composition comprising one or more compounds of formula (I) and a product base.

By “product base” is meant the combination of all the usual art-recognized ingredients required for the particular consumable composition.

In another aspect of the present invention, there is provided a consumer product selected from the group consisting of foodstuffs and beverages, said consumer product comprising at least 1 ppm, preferably at least 20 ppm, more preferably at least 50 ppm or 70 ppm ppb of one or more flavor modulating compounds according to formula (I) and/or edible salts thereof.

For example, said product contains at least 0.0001 wt. %, more preferably at least 0.0003 wt. %, even more preferably at least 0.001 wt. %, most preferably at least 0.003 wt. % of the one or more flavor modulating compounds. Typically, the aforementioned products will contain the flavor modulating compounds in a concentration of not more than 1 wt. %, preferably of not more than 0.5 wt. %.

Typical examples of foodstuffs according to the present invention include soups, sauces, stocks, bouillons, broths, cheese products, for example cheese sauces, vegan cheese alternatives, dressings, mayonnaise, seasonings, margarines, noodles, chips, curls, meat products, vegan meat alternatives and beverages. For example, the mentioned foodstuff can be a low sodium product.

The flavor modulating compounds according to the present invention can be applied advantageously to impart desirable taste attributes to the aforementioned products. In addition, the present taste improving substances are capable of modulating the taste impact of other flavor ingredients contained within these same products, thereby improving the overall flavor quality of these products.

In another aspect of the present invention, there is provided the use of the compounds of formula (I) as flavor modulating compounds.

The compounds can be used in pure form or in form of diluted form, provided in liquid or in solid form.

In another aspect of the present invention, there is provided a method to confer, enhance, improve or modify the flavor properties of a flavor composition or a consumer product comprising adding to said composition or consumer product at least one flavor modulating compound, which is the compound of formula (I) or an edible salt thereof. For example, the flavor modulating compound is added in an amount of at least 0.0003 wt. %, preferably of at 20) least 0.001 wt. %.

Some of the compounds of formula (I) are known from different application, however, most of the compounds are novel.

For example, 3-(1H-imidazol-4-yl)-N-[2-(1H-imidazol-4-yl)ethyl]prop-2-enamide was described by Baures et al (Molecules (2002), 7(11), 813-816) as a natural histamine derivative. No organoleptic properties of said compound were disclosed.

Therefore, the invention provides a compound of formula (I)

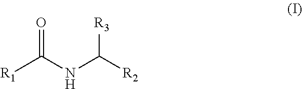

and edible salts thereof, wherein

R₁ is selected from the group consisting of 2-(1H-4-imidazolyl)-ethenyl, 1H-5-indolyl, 2-(1H-5-imidazolyl)-ethenyl, 1-amino-2-(1H-4-imidazolyl)-ethyl, (1,3-thiazol-2-yl)-ethenyl, 2,3-dihydro-1H-indol-2-yl, 2-(pyrimidin-2-yl)ethenyl, heptadecanyl, 1-heptadec-8-enyl, heptadeca-8,11-dienyl, heptadeca-8,11,14-trienyl, 2-(4H-imidazol-2-yl)-ethenyl, 2-(4H-imidazol-2-yl)-ethyl, 2-phenyl-ethenyl, 2-(furan-2-yl)-ethenyl, 2-(thiophen-2-yl)-ethenyl, 2-(thiophen-3-yl)-ethenyl, 2but-2-enoyl, 2-butyl, 2,6-dimethylhepta-1,5-dienyl, 1,3-benzothiazol-6-yl;

R₂ is selected from the group consisting of (1H-imidazol-4-yl)-methyl, (1H-3-indol-3-yl)-methyl, 4-hydroxybenzyl, methylsulfanylethyl, hydroxymethyl, CH₂-COOH, (pyridin-4-yl)methyl, (pyridin-2-yl)methyl, 1-(2,6-dimethylhepta-1,5-dienyl), 2-(pyrimidin-2-yl)methyl, (1H-imidazol-5-yl)-methyl, (1H-imidazol-2-yl)-methyl, (1H-pyrrol-2-yl)-methyl, phenyl, pyrimidin-5-yl, pyrazin-2-yl;

R₃ is selected from the group consisting of H, COOH,

with the proviso that the compound is not 3-(1H-imidazol-4-yl)-N-[2-(1H-imidazol-4-yl)ethyl]prop-2-enamide, histidyltyrosine, histidylhistidine, N-(2-hydroxyethyl)histidinamide, histidyl-B-alanine, N-octadecanoylhistidine, N-[(9Z)-octadec-9-enoyl]histidine.

In a further aspect, there is provided a compounds of formula (I) and edible salts thereof as flavor modulating compounds, as defined above, wherein the compound possess at least one aromatic unit attached to a residue via an amide bond. For example, the aromatic unit can be selected from the group consisting of phenyl, imidazole, thiazole, indole, furan, thiophen, benzothiazol, pyrimidine, pyrazine, pyrrol. The aromatic unit can be a moiety of R₁ and/or R₂.

In one embodiment, the aromatic unit is imidazole.

In a further aspect, there is provided a compound of formula (I) and edible salts thereof as flavor modulating compounds, as defined above, wherein the compound possess two aromatic units attached to a residue via an amide bond, and wherein the aromatic unit can be a moiety of R₁ and R₂. For example, the aromatic unit can be selected from the group consisting of phenyl, imidazole, thiazole, indole, furan, thiophen, benzothiazol, pyrimidine, pyrazine, pyrrol.

In one embodiment, at least one of the two aromatic units attached to a residue via an amide bond is imidazole.

In a further aspect, there is provided the compound of formula (I) and edible salts thereof as flavor modulating compound, wherein the compound is represented by formula (II) in the form of any one of its isomers or a mixture thereof

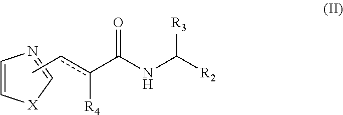

wherein

[symbol] is indicating a carbon-carbon single or double bond,

X is representing a heteroatom selected from the group consisting of N and S,

R₄ is either H or NH₂,

and R₂ and R₃ have the same meaning as defined for the compound of formula (I).

The compounds of formula II possess a five membered unsaturated heterocycle with two heteroatoms attached to a residue via an amide bond. For example, the five membered unsaturated heterocycle is imidazole or thiazole. In a further aspect, there is provided a compound of formula (I) and edible salts thereof as flavor modulating compounds, wherein the compound is represented by formula (III) in the form of any one of its isomers or a mixture thereof,

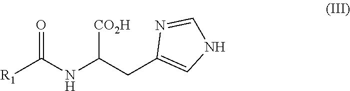

wherein R₁ is selected from the group consisting of heptadecanyl, 1-heptadec-8-enyl, heptadeca-8,11-dienyl and heptadeca-8,11,14-trienyl.

In a further aspect, the invention provides a compound of formula (I) as defined above, wherein the compound is selected from the group consisting of N-[2-(1H-imidazol-4-yl) ethyl]-1H-indole-5-carboxamide, N-[3-(1H-imidazol-5-yl)prop-2-enoyl]tryptophan, N-[3-(methylsulfanyl)propyl]histidinamide, N-[octadeca-9,12-dienoyl]histidine, N-[2-(1H-imidazol-4-yl)ethyl]-3-(1,3-thiazol-2-yl)prop-2-enamide, N-[octadeca-9,12, 15-trienoyl]histidine, 3-(1H-imidazol-4-yl)-N-[2-(pyridin-4-yl) ethyl]prop-2-enamide, 3-(1H-imidazol-4-yl)-N-[2-(pyridin-2-yl)ethyl]prop-2-enamide, N-[3,7-dimethylocta-2,6-dien-1-yl]-3-(1H-imidazol-4-yl) prop-2-enamide, N-[2-(1H-imidazol-4-yl) ethyl]-2,3-dihydro-1H-indole-2-carboxamide, 3-(pyrimidin-2-yl)-N-[2-(pyrimidin-2-yl)ethyl]prop-2-enamide, 3-(4H-imidazol-2-yl)-N-[2-(1H-imidazol-5-yl)ethyl]prop-2-enamide, 3-(1H-imidazol-4-yl)-N-[2-(1H-imidazol-2-yl) ethyl]prop-2-enamide, N-(2-hydroxyethyl)-3-(1H-imidazol-4-yl)propanamide, 3-(1H-imidazol-4-yl)-N-[2-(1H-pyrrol-2-yl)ethyl]prop-2-enamide, N-benzyl-3-(1H-imidazol-4-yl)prop-2-enamide, 3-(1H-imidazol-4-yl)-N-[(pyrimidin-5-yl)methyl]prop-2-enamide, 3-(1H-imidazol-4-yl)-N-[(pyrazin-2-yl)methyl]prop-2-enamide, 3-(furan-2-yl)-N-[2-(1H-imidazol-4-yl)ethyl]prop-2-enamide, N-[2-(1H-imidazol-4-yl)ethyl]-3-(thiophen-2-yl)prop-2-enamide, N-[2-(1H-imidazol-4-yl)ethyl]-3-(thiophen-3-yl)prop-2-enamide, 3-(1H-imidazol-4-yl)-2-{[(2E)-2-methylbut-2-enoyl]amino}propanoic acid, N-[2-(1H-imidazol-4-yl)ethyl]-2-methylbutanamide, N-[2-(1H-imidazol-4-yl)ethyl]-3,7-dimethylocta-2,6-dienamide, 2-{[3-(furan-2-yl)prop-2-enoyl]amino}-3-(1H-imidazol-4-yl)propanoic acid, 3-(1H-imidazol-5-yl)-2-{[3-(1H-imidazol-4-yl)prop-2-enoyl]amino}propanoic acid, N-[2-(1H-imidazol-5-yl)ethyl]-1,3-benzothiazole-6-carboxamide, 3-(1H-imidazol-4-yl)-N-(2-phenylethyl)prop-2-enamide.

The compounds of the present invention may be prepared from amino acids or modified amino acids, like decarboxylated or desaminated amino acids and from fatty acids.

For example, the compounds of the present invention may be prepared from amino acids selected from the group consisting of B-Alanine, Histidine, Tryptophan, and Tyrosine, from modified amino acids selected from the group consisting of decarboxylated Histidine, Serine, Methionine, Tyrosine, or desaminated and dehydrogenated Histidine.

The compounds of the present invention may be prepared from two amino acids by peptide synthesis. They are suitably produced by reacting an amine of a first amino acid with a carboxyl group of a second amino acid.

Other compounds of the present invention may be prepared from one amino acid or its derivative and a fatty acid.

The preparation of the compounds of the present invention can be carried out by methods known in the art. For example, the compounds can be obtained by chemical or enzymatic reactions.

The invention is now further described with reference to the following non-limiting examples. These examples are for the purpose of illustration only and it is understood that variations and modifications can be made by one skilled in the art.

### EXAMPLES

### Example 1: (2E)-3-(1H-imidazol-4-yl)-N-[2-(1H-imidazol-4-yl)ethyl]prop-2-enamide

(E)-3-(1H-imidazol-4-yl)acrylic acid (13.8 g, 100 mmol) was dissolved in DMF (800 ml). 1-hydroxypyrrolidine-2,5-dione (12.66 g, 110 mmol) and dicyclohexylmethanediimine (22.70 g, 110 mmol) were added while stirring at room temperature. Stirring was continued for 24 hours, and the solvent was evaporated till a volume of approx. 200 mL. Solids (dicyclohexylurea) were filtered to obtain 220 g of filtrate.

To the filtrate (DMF solution of 2,5-dioxopyrrolidin-1-yl (E)-3-(1H-imidazol-4-yl)acrylate) was added an aqueous solution of Histamine.2HCl (18.4 g, 100 mmol) and sodiumbicarbonate (16.8 g, 200 mmol) and the mixture was stirred at 50° C. for 3 hours. Solvent was evaporated and methanol (200 mL) was added to the residue. Remaining solids (NaCl) were filtered and filtrate was concentrated at reduced pressure. The residue was purified by flash column chromatography. The isolated product was stirred three hours with acetone to remove the impurity NHS. Product was filtered and dried in the vacuum oven at 50° C./15 mbar. 15 g of (2E)-3-(1H-imidazol-4-yl)-N-[2-(1H-imidazol-4-yl)ethyl]prop-2-enamide was obtained in a purity of 95% as a white powder. Yield: 62%

¹H NMR (D₂O, 600 MHz) δ=8.35 (1H, s), 7.83 (1H, s), 7.44 (1H, s), 7.42 (1H, d, J=15.1 Hz), 7.19 (1H, s), 6.49 (1H, d, J=15.8 Hz), 3.61 (2H, J=6.5 Hz), 2.96 (2H, J=6.5 Hz). ¹³C NMR (D₂O, 151 MHz) δ=171.9, 140.9, 137.7, 136.7, 134.8, 124.4, 120.0, 119.3, 41.4, 27.6.

### Example 2: N-[2-(1H-imidazol-4-yl)ethyl]-1H-indole-5-carboxamide

1H-indole-5-carboxylic acid (5 g, 31.0 mmol) was dissolved in DMF (350 mL) 1-hydroxypyrrolidine-2,5-dione (3.93 g, 34.1 mmol) and dicyclohexylmethanediimine (7.04 g, 34.1 mmol) were added while stirring at room temperature. Stirring was continued for 24 hours. The solids were filtrated, and filtrate was evaporated till approx. 100 mL.

To the DMF solution of 2,5-dioxopyrrolidin-1-yl 1H-indole-5-carboxylate (8.01 g, 31 mmol) was added a solution of sodium bicarbonate (5.21 g, 62.0 mmol) and Histamine. 2HCl in Water (50 ml). This mixture was stirred at 50° C. for 4 hours. Volatiles were evaporated and the residue was taken up in methanol. The remaining solids (NaCl) were filtered and filtrate was evaporated. The residue was purified by flash column chromatography with eluent DCM/Methanol. The isolated product was further purified with acid Dowex cation exchanger to remove the remaining NHS, which resulted finally in 200 mg of 90% pure product.

¹H NMR (DMSO-d₆, 600 MHz) δ=8.45 (1H, t, J=5.5 Hz), 8.11 (1H, s), 7.69-7.79 (1H, m), 7.62 (1H, d, J=8.0 Hz), 7.40-7.49 (2H, m), 6.93 (1H, s), 6.52 (1H, br s), 3.38-3.63 (2H, m), 2.80 (2H, t, J=7.6 Hz). ¹³C NMR (DMSO-d₆, 151 MHz) δ=167.0, 137.1, 134.3, 133.9, 126.8, 126.4, 125.4, 120.2, 119.6, 116.5, 110.6, 101.9, 59.6, 39.1, 26.5.

### Example 3: L-Histidyl-L-tyrosine

The compound was obtained from Bachem.

¹H NMR (D₂O, 600 MHz) δ=7.99 (1H, s), 7.11-7.17 (2H, m, J=8.3 Hz), 7.08 (1H, s), 6.81-6.87 (2H, m), 4.40 (1H, dd, J=8.6 Hz, J=5.2 Hz), 3.99-4.05 (1H, m), 3.08-3.17 (3H, m), 2.91 (1H, dd, J=14.1 Hz, J=8.6 Hz). ¹³C NMR (D₂O, 151 MHz) δ=180.7, 173.2, 157.0, 138.5, 133.4, 132.5, 132.3, 120.6, 118.2, 59.6, 56.0, 39.3, 31.6.

### Example 4: N-[(2E)-3-(1H-imidazol-5-yl)prop-2-enoyl]-L-tryptophan

4 g of methyl L-tryptophanate was solved in DMF and while stirring an amount of 1.54 g of NaHCO3 was added. Then 175 ml of a solution of freshly prepared 0.125 mol/l of 2,5-dioxopyrrolidin-1-yl 3-(1H-imidazol-4-yl) acrylate in DMF was added. The obtained mixture was warmed to 40° C. and stirring continued for 6 hrs. The reaction mixture was concentrated by evaporating the DMF. To the residue was added 250 ml of ethyl acetate and washed with 100 ml water. The ethyl acetate extract was washed again with an amount of 100 ml of water and concentrated by evaporation. Purification was done using flash chromatography with dichloromethane: methanol. Obtained was 3.5 g of the intermediate methyl (E)-(4-(1H-imidazol-5-yl)but-2-enoyl)-L-tryptophanate.

Subsequently the obtained methyl ester was hydrolyzed using the following procedure: 1 g of methyl (E)-(4-(1H-imidazol-5-yl)but-2-enoyl)-L-tryptophanate was solved in methanol. To the solution was added 12 ml of a 1 M NaOH solution. After completion of the hydrolysis, the mixture was cooled to 0° C. and acidified to pH 1.8 with a 1 M HCl solution.

The obtained mixture was concentrated by evaporation, and ethyl acetate was added to the residue. After filtration, the mixture was concentrated again by evaporation and purified by flash chromatography with dichloromethane and methanol. Obtained was 0.5 gram of the desired product.

¹H NMR (DMSO-d₆, 600 MHz) δ=10.92 (1H, s), 9.13 (1H, s), 8.70 (1H, d, J=7.6 Hz), 7.93 (1H, s), 7.55 (1H, d, J=8.3 Hz), 7.33 (2H, br d, J=8.3 Hz), 7.31 (2H, br d, J=15.8 Hz), 7.16 (1H, d, J=2.1 Hz), 7.05 (1H, t, J=7.6 Hz), 6.98 (1H, t, J_{5,6}=7.2 Hz), 6.72 (1H, d, J=15.8 Hz, 4.54-4.63 (1H, m), 3.20-3.28 (1H, m), 3.09 (1H, dd, J=14.8 Hz, J=9.3 Hz). ¹³C NMR (DMSO-d₆, 151 MHz) δ=173.4, 164.1, 136.1, 135.9, 129.4, 127.1, 123.6, 121.0, 119.9, 118.4, 118.2, 111.5, 110.0 48.6, 40.0, 26.9

### Example 5: L-Histidyl-L-histidine

The compound was obtained from Bachem.

¹H NMR (DMSO-d₆, 600 MHz) δ=9.15 (1H, br d, J=7.6 Hz), 8.96 (1H, s), 8.79 (1H, br s,), 7.44 (1H, s), 7.37 (1H, s), 4.54-4.61 (1H, m), 4.24 (1H, t, J=6.5 Hz), 3.14-3.26 (3H, m), 3.07 (1H, dd, J=15.1 Hz, J=9.0 Hz). ¹³C NMR (DMSO-d₆, 151 MHz) δ=171.5, 167.6, 134.5, 133.9, 129.3, 127.7, 118.1, 117.6, 117.1, 116.2, 60.4, 52.0, 51.3, 26.7, 26.1.

### Example 6: N-[3-(methylsulfanyl)propyl]histidinamide

a) To a mixture of N₆₀ -Boc-histidine (3.0 g, 11.75 mmol) and 1-hydroxypyrrolidine-2,5-dione (1.62 g, 14.10 mmol) in DMF (100 mL), dicyclohexylmethanediimine (2.91 g, 14.10 mmol) was added and stirred overnight at room temperature. The formed dicyclohexylurea was filtered off. To the filtrate, 3-(methylthio)propan-1-amine (1.5 g, 14.10 mmol) was added and stirred for 3 hours at 50° C. After removal of DMF by evaporation under reduced pressure, the residue was taken in 100 ml water and then extracted twice with ethyl acetate (2× 100 ml). The organic was washed once with brine (100 ml), dried over magnesium sulfate, filtered and evaporated. Purification of the crude product by silica gel column chromatography using DCM/methanol resulted in the intermediate, 3-(methylthio)propanyl-N_{α}-Boc-histidinamide as off white solid.

b) The intermediate 3-(methylthio)propanyl-N_{α}-Boc-histidinamide (0.36 g, 1.051 mmol) was dissolved in 100 ml methanol and cooled with an ice bath to 4° C. Then 15 ml 3 M HCl in methanol was added and allowed to stir for 2 hours. The solvent was evaporated and the residue was purified by silica gel column using DCM/methanol to yield 0.15 g (48.6%) of the target N-[3-(methylsulfanyl)propyl]histidinamide hydrochloride as pink solid. Purity is >95% by NMR analysis.

¹H NMR (DMSO-d₆, 600 MHz) δ=9.08 (1H, s), 8.82 (1H, t, J=5.5 Hz), 8.55 (3H, br s), 7.51 (1H, s), 4.22 (1H, br t, J=6.2 Hz), 3.31-3.37 (1H, m), 3.11-3.26 (4H, m), 2.42 (2H, td, J=7.2 Hz, J=3.4 Hz), 2.02 (3H, s), 1.57-1.68 (2H, m). ¹³C NMR (DMSO-d₆, 151 MHz) δ=167.0, 134.0, 127.0, 117.7, 65.0, 51.3, 37.7, 30.4, 28.1, 26.4, 14.6.

### Example 7: N-(2-hydroxyethyl)histidinamide

N-(2-hydroxyethyl) histidinamide was prepared by the procedure of example 6. Boc-His-OH (3.0 g, 11.75 mmol) was coupled with ethanolamine (0.86 g, 14.10 mmol) by using dicyclohexylmethanediimine (2.91 g, 14.10 mmol) and 1-hydroxypyrrolidine-2,5-dione (1.62 g, 14.10 mmol). The target N-(2-hydroxyethyl)histidinamide hydrochloride was obtained as white precipitate by addition of ether to the reaction mixture in step 2. Yield: 0.7 g (22.5%); Purity is >95% by NMR analysis.

¹H NMR (DMSO-d₆, 600 MHz) δ=9.04 (1H, s), 8.67 (1H, br d, J=4.8 Hz), 7.46 (1H, s), 4.14 (1H, br t, J=6.5 Hz), 3.30-3.48 (2H, m), 3.17-3.26 (2H, m), 3.11-3.17 (2H, m, H-1), 3.06-3.11 (2H, m). ¹³C NMR (DMSO-d₆, 151 MHz) δ=167.3, 134.2, 126.9, 118.2, 59.5, 51.5, 42.0, 26.4.

### Example 8: Histidyl-B-alanine

Histidyl-β-alanine hydrochloride was prepared by procedure as described for example 6. Boc-His-OH (3.0 g, 11.75 mmol) was coupled with β-alanine (1.25 g, 14.10 mmol) by using coupling reagents dicyclohexylmethanediimine (2.91 g,, 14.10 mmol) and 1-hydroxypyrrolidine-2,5-dione (1.62 g, 14.10 mmol). 0.84 g of the target compound was yielded as white solid. Purity is >95% by NMR analysis.

¹H NMR (D₂O, 600 MHz) δ=8.56 (1H, s), 7.38 (1H), 4.22 (1H, t, J=6.5 Hz), 3.46-3.55 (1H, m), 3.31-3.37 (1H, m), 3.29-3.40 (3H, m), 2.36 (2H, td, J=6.5 Hz, J=2.1 Hz). ¹³C NMR (D₂O, 151 MHz) δ=182.7, 170.9, 137.6, 129.6, 121.1, 55.3, 51.8, 39.6, 39.0, 29.5.

### Example 9: N-[(9Z,12Z)-octadeca-9,12-dienoyl]-L-histidine

L-histidine hydrochloride (4.13 g, 21.54 mmol) was dissolved in 60 ml of aqueous NaOH (2.37 g, 59.2 mmol) solution. The solution was diluted with 60 ml THF and cooled with an ice bath. Then a solution of (9Z,12Z)-octadeca-9,12-dienoyl chloride (5.33 g, 17.95 mmol) in 40 ml THF was added dropwise. After 2 hours stirring at room temperature, the reaction mixture was acidified with dilute HCl solution and then extracted with 200 ml ethyl acetate. The formed precipitate in ethyl acetate was filtered, washed with hot heptane and then dried in a vacuum oven at 40° C. 2.96 g of ((9Z, 12Z)-octadeca-9,12-dienoyl)-L-histidine was yielded as orange solid. Purity is >95% by NMR analysis.

¹H NMR (DMSO-d₆, 600 MHz) δ=8.54 (1H, s), 8.22 (1H, d, J=8.3 Hz), 7.18 (1H, s), 5.24-5.40 (3H, m), 4.48 (1H, td, J=8.8 Hz, J=5.2 Hz), 3.06 (1H, dd, J=15.1 Hz, J=4.8 Hz), 2.93 (1H, dd, J=14.8 Hz, J=9.3 Hz), 2.73 (1H, br t, J=6.9 Hz), 2.06 (2H, br t, J=7.2 Hz), 1.92-2.03 (4H, m, H-8), 1.44-1.52 (1H, m), 1.41 (2H, quin, J=7.4 Hz), 1.09-1.35 (16H, m), 0.85 (3H, br t, J=6.9 Hz). ¹³C NMR (DMSO-d₆, 151 MHz) δ=172.6, 172.3, 133.9, 130.8, 129.8, 127.8, 116.8, 51.4, 40.0, 35.1, 31.3, 30.9, 29.1, 29.1, 28.9, 28.8, 28.7, 28.6, 28.5, 27.0, 26.7, 26.6, 25.2, 25.2, 22.1, 22.0, 14.0.

### Example 10: (2E)-N-[2-(1H-imidazol-4-yl)ethyl]-3-(1,3-thiazol-2-yl)prop-2-enamide

(E)-3-(thiazol-2-yl) acrylic acid (1 g, 6.44 mmol) was dissolved in DMF (25 ml) under heating. Di(1H-imidazol-1-yl) methanone (1.254 g, 7.73 mmol) was added while stirring, and the reaction mixture was stirred during 1 day at RT. TEA (0.898 ml, 6.44 mmol) and 2-(1H-imidazol-4-yl)ethan-1-amine (0.716 g, 6.44 mmol) were added, and stirring was continued for 1 day at RT. Then, the mixture was stirred at 50° C. for 3 hours. The solvent was evaporated. The solid residue was taken up in acetone and filtered. Solids were further purified by flash column chromatography. 0.3 g of light brown product were obtained. Purity is >95% by NMR analysis.

¹H NMR (D₂O, 600 MHz) δ=8.62 (1H, s), 8.23 (1H, d, J=3.3 Hz), 8.12 (1H, d, J=4.1 Hz, H-5), 7.70 (1H, d, J=15.8 Hz), 7.30 (1H, s), 7.13 (1H, d, J_{3,2(E)}=15.8 Hz), 3.67 (2H, t, J=6.5 Hz), 3.04 (2H, t, J_{2,1}=6.5 Hz). ¹³C NMR (D₂O, 151 MHz) δ=169.1, 168.2, 138.3, 136.1, 135.3, 133.5, 128.0, 127.9, 119.3, 41.5, 26.9.

### Example 11: N-[(9Z,12Z,15Z)-octadeca-9,12,15-trienoyl]-L-histidine

N-[(9Z,12Z,15Z)-octadeca-9,12,15-trienoyl]-L-histidine was prepared according to the procedure of example 9. L-Histidine hydrochloride (4.13 g, 21.54 mmol) was reacted with (9Z,12Z,15Z)-octadeca-9,12,15-trienoyl chloride (5.33 g, 17.95 mmol) to obtain 2.33 g of N-[(9Z,12Z,15Z)-octadeca-9,12,15-trienoyl]-L-histidine as orange solid.

¹H NMR (DMSO-d₆, 600 MHz) δ=7.97 (1H, br d, J=7.6 Hz), 5.26-5.41 (5H, m), 4.41-4.53 (1H, m), 3.01 (1H, dd, J=14.8 Hz, J=5.2 Hz), 2.89 (1H, dd, J=14.8 Hz, J=8.6 Hz), 2.72-2.81 (3H, m, H-11), 1.99-2.11 (5H, m), 1.38-1.51 (2H, m), 1.17-1.34 (10H, m), 0.93 (2H, t, J=7.6 Hz), 0.82-0.89 (1H, m). ¹³C NMR (DMSO-d₆, 151 MHz) δ=172.5, 171.9, 134.1, 131.3, 129.8, 129.6, 127.8, 127.6, 127.3, 126.8, 116.6, 51.6, 40.0, 35.0, 30.7, 28.8, 28.5, 28.4, 28.3, 27.9, 26.5, 26.4, 25.0, 24.9, 24.3, 21.7, 19.8, 13.8, 13.6.

### Example 12: N-[(9Z)-octadec-9-enoyl]-L-histidine

A solution of (Z)-docos-13-enoyl chloride (11.69 g; 32.7 mmol) in 30 ml DCM was added dropwise to a mixture of ethyl L-histdinate (5 g; 27.3 mmol) and triethylamine (11.05 g; 109 mmol) in 300 ml DCM. After 2 hours stirring at room temperature, the reaction mixture was washed with water (2×150 ml). The DCM layer was dried with MgSO4 and then evaporated under reduced pressure to obtain the intermediate ethyl oleoyl-L-histidinate.

The hydrolysis of this intermediate was carried out by addition of solution of sodium hydroxide (1.56 g; 39.0 mmol) in 50 ml water to a cold solution of ethyl oleoyl-L-histidinate (8.74 g; 19.52 mmol) in 50 ml methanol (50 ml). After 2 hours stirring at room temperature, the reaction mixture was acidified with diluted HCl. The formed solid was filtered off, washed with heptane and n-pentane and then dried in vacuum oven at 40° C. 0.3 g (3%) of oleoyl-L-histidine was yielded as white solid. Purity is >95% by NMR analysis.

¹H NMR (DMSO-d₆, 600 MHz) δ=8.03 (1H, br d, J=7.6 Hz), 7.55 (1H, s), 6.78 (1H, s), 5.27-5.36 (2H, m), 4.32-4.44 (1H, m), 2.91 (1H, br dd, J_{>}=15.1 Hz, J=4.8 Hz), 2.81 (1H, br dd, J=14.8 Hz, J=8.6 Hz), 2.01-2.09 (2H, m), 1.95-2.00 (3H), 1.42 (3H, dt, J=14.1 Hz, J=7.4 Hz), 1.10-1.34 (21H, m), 0.84 (3H, br t, J=6.9 Hz, H-18). ¹³C NMR (DMSO-d₆, 151 MHz) δ=172.8, 171.8, 134.3, 129.4, 129.4, 52.0, 40.0, 35.0, 31.0, 28.9, 28.9, 28.8, 28.7, 28.6, 28.4, 28.4, 28.4, 28.3, 26.4, 26.4, 24.9, 21.8, 13.6.

### Example 13: N-octadecanoylhistidine

N-octadecanoylhistidine was prepared according to the procedure of example 9. L-Histidine hydrochloride (3.77g, 19.69 mmol) was reacted with stearoyl chloride (4.97 g; 16.41 mmol) to obtain 1.9 g (26%) of the target compound as white solid. Purity is >95% by NMR analysis. ¹H NMR (DMSO-d₆, 600 MHz) δ=8.11 (1H, d, J=7.6 Hz), 8.04 (1H, br s), 7.93 (1H, s), 7.06 (1H, s), 6.98 (1H, s), 4.44 (1H, td, J=8.3 Hz, J=5.5 Hz), 3.86-3.91 (2H, m), 3.02-3.09 (2H, m), 2.97 (3H, td, J=14.8 Hz, J=6.2 Hz), 2.86 (2H, br dd, J=15.1 Hz, J=9.0 Hz), 2.18 (1H, t, J=7.6 Hz), 2.05 (2H, t, J=7.6 Hz), 1.44-1.50 (1H, m), 1.41 (2H, quin, J=7.4 Hz), 1.24-1.28 (4H, m), 1.23 (28H, s), 1.15-1.18 (2H, m), 0.85 (4H, t, J=6.9 Hz). ¹³C NMR (DMSO-d₆, 151 MHz) δ=174.6, 172.9, 172.2, 170.0, 134.8, 134.4, 52.8, 51.7, 40.0, 35.1, 33.7, 31.3, 29.1, 29.1, 29.0, 28.8, 28.8, 28.6, 28.7, 27.2, 25.2, 24.5, 22.1, 14.0.

### Example 14: (2E)-3-(1H-imidazol-4-yl)prop-2-enamide

The compound was obtained from Aldrich.

¹H NMR (DMSO-d₆, 600 MHz) δ=11.89-12.77 (1H), 7.71 (1H, s), 7.37-7.52 (1H), 7.36 (1H, s), 7.28 (1H, d, J=15.8 Hz), 6.90 (1H, br s), 6.47 (1H, br d, J=15.8 Hz). ¹³C NMR (DMSO-d₆, 151 MHz) δ=167.4, 137.1, 131.3, 118.5.

### Example 15: (2E)-3-(1H-imidazol-4-yl)-N-[2-(pyridin-4-yl)ethyl]prop-2-enamide

A mixture of (E)-3-(1H-imidazol-4-yl)acrylic acid (2.1 g, 15.20 mmol), 1-hydroxypyrrolidine-2,5-dione (1.925 g, 16.72 mmol) and DCC (3.45 g, 16.72 mmol) in DMF (150 ml) was stirred for 24 hours. Then the formed dicyclohexylurea was filtered off. To the filtrate, 2-(pyridin-4-yl)ethan-1-amine (2 g, 16.37 mmol) was added and stirred for 4 hours at 50° C. After removal of DMF by evaporation under reduced pressure, the remaining residual crude product was added to a silica gel column and eluted with DCM/methanol. 0.9 g (24.4%) of (2E)-3-(1H-imidazol-4-yl)-N-[2-(pyridin-4-yl)ethyl]prop-2-enamide was yielded as white solid. Purity is >95% by NMR.

¹H NMR (DMSO-d₆, 600 MHz) δ=8.28-8.64 (2H, m), 8.12 (1H, br s), 7.69 (1H, br s), 7.39 (1H, br s), 7.28 (1H, d, J=15.1 Hz), 7.24 (2H, d, J=5.5 Hz), 6.50 (1H, br d, J=16.5 Hz), 3.42 (2H, q, J=6.9 Hz), 2.78 (2H, t, J=6.9 Hz). ¹³C NMR (DMSO-d₆, 151 MHz) δ=165.8, 149.5, 148.5, 137.2, 136.9, 131.7, 124.3, 118.9, 118.2, 40.0, 34.4.

### Example 16: methyl 2,3-dihydro-1H-indole-2-carboxylate

Indoline-2-carboxylic acid (5 g, 30.6 mmol) was dissolved in 100 ml methanol and cooled with an ice bath. While stirring, acetyl chloride (16.5 g, 210 mmol) was added dropwise. After stirring for 1 hr in an ice bath, the solution was allowed to stand at room temperature overnight. Then the solvent was removed by evaporation under reduced pressure at 30° C. The remaining residual solid was recrystallized from methanol to yield 6.3g (96%) of methyl indoline-2-carboxylate, HCl as white solid. Purity is >95% by NMR.

¹H NMR (DMSO-d₆, 600 MHz) δ=7.11 (1H, d, J=7.6 Hz), 7.04 (1H, t, J=7.6 Hz), 6.73-6.80 (2H, m), 4.54 (1H, dd, J=10.0 Hz, J=6.5 Hz), 3.65-3.71 (3H, m), 3.35 (1H, dd, J=16.5 Hz, J=10.3 Hz), 3.15 (1H, dd, J=15.8 Hz, J=6.2 Hz). ¹³C NMR (DMSO-d₆, 151 MHz) δ=172.7, 147.4, 127.9, 127.4, 124.4, 120.1, 111.0, 59.0, 52.1, 32.9.

### Example 17: (2E)-3-(1H-imidazol-4-yl)-N-[2-(pyridin-2-yl)ethyl]prop-2-enamide

(2E)-3-(1H-imidazol-4-yl)-N-[2-(pyridin-2-yl)ethyl]prop-2-enamide was prepared according to the procedure of example 15. (E)-3-(1H-imidazol-4-yl) acrylic acid (2 g, 14.48 mmol) was coupled with 2-(pyridin-2-yl)ethan-1-amine (2 g, 16.37 mmol) using 1-hydroxypyrrolidine-2,5-dione (1.833 g, 15.93 mmol) and DCC (3.29 g, 15.93 mmol). 0.9 g (25.7%) of (2E)-3-(1H-imidazol-4-yl)-N-[2-(pyridin-2-yl)ethyl]prop-2-enamide was yielded as white solid. Purity is >95% by NMR.

¹H NMR (DMSO-d₆, 600 MHz) δ=9.19 (1H, br s), 8.78-8.83 (1H, m), 8.75-8.78 (1H, m), 8.50 (1H, t, J=7.8 Hz), 7.96 (1H, br d, J=8.3 Hz), 7.94 (1H, s), 7.92 (1H, t, J=6.9 Hz), 7.26 (1H, dt, J=15.8 Hz, J=1.4 Hz), 6.71 (1H, d, J=15.8 Hz), 5.35 (7H, br s), 3.63 (2H, br t, J=6.2 Hz), 3.26 (2H, br t, J=6.2 Hz). ¹³C NMR (DMSO-d₆, 151 MHz) δ=164.6, 154.5, 146.6, 141.3, 135.8, 129.1, 128.0, 125.5, 124.6, 124.4, 120.1, 38.2, 33.0.

### Example 18: (2E)-N-[(2E)-3,7-dimethylocta-2,6-dien-1-yl]-3-(1H-imidazol-4-yl)prop-2-enamide

(2E)-N-[(2E)-3,7-dimethylocta-2,6-dien-1-yl]-3-(1H-imidazol-4-yl)prop-2-enamide was prepared according to the procedure of example 15. (E)-3-(1H-imidazol-4-yl)acrylic acid (2 g, 14.48 mmol) was coupled with Geranyl amine (2.441 g, 15.93 mmol) using 1-hydroxypyrrolidine-2,5-dione (1.833 g, 15.93 mmol) and DCC (3.29 g, 15.93 mmol). 1.1 g (27.8%) of (2E)-N-[(2E)-3,7-dimethylocta-2,6-dien-1-yl]-3-(1H-imidazol-4-yl)prop-2-enamide was yielded as off white solid. Purity is >90% by NMR.

¹H NMR (CDCl₃, 600 MHz) δ=9.46 (1H, br s), 7.82 (1H, s), 7.42 (1H, d, J=15.1 Hz), 7.15 (1H, s), 6.88 (1H, br s), 6.60 (1H, d, J=15.1 Hz, 5.17-5.25 (1H, m), 4.97-5.13 (1H, m), 3.92 (2H, br t, J=6.2 Hz), 2.00-2.07 (2H, m), 1.94-2.00 (2H, m), 1.65 (3H, br s), 1.64 (3H, br s), 1.56 (3H, s). ¹³C NMR (CDCl₃, 151 MHz) δ=166.3, 139.8, 136.5, 133.8, 131.7, 129.5, 123.8, 121.4, 119.8, 119.6, 39.5, 39.4, 37.8, 25., 16.3.

### Example 19: N-[2-(1H-imidazol-4-yl)ethyl]-2,3-dihydro-1H-indole-2-carboxamide

A solution of methyl indoline-2-carboxylate (1.34 g, 7.56 mmol) and histamine (0.6 g, 5.40 mmol) in 20 ml THF and 20 ml methanol) was stirred at reflux for 4 hrs. After removal of the solvents, the crude product was purified by silica gel column chromatography with DCM and methanol. 0.3 g (20.6%) of N-[2-(1H-imidazol-4-yl)ethyl]-2,3-dihydro-1H-indole-2-carboxamide was yielded as white solid. Purity is >95% by NMR.

¹H NMR (DMSO-d₆, 600 MHz) δ=7.97 (1H, t, J=5.5 Hz), 7.60 (1H, s), 6.99 (1H, d, J=7.6 Hz), 6.94 (1H, t, J_{6,4}=7.6 Hz), 6.81 (1H, s), 6.55-6.60 (2H, m), 5.93 (1H, br s), 4.18 (1H, dd, J=10.3 Hz, J=8.3 Hz), 3.29-3.37 (2H, m), 3.27 (1H, dd, J=15.8 Hz, J=9.6 Hz), 2.86 (1H, dd, J=15.8 Hz, J=8.3 Hz), 2.65 (2H, t, J=7.2 Hz). ¹³C NMR (DMSO-d₆, 151 MHz) δ=173.3, 151.2, 134.6, 127.3, 127.2, 124.2, 117.9, 116.5, 109.1, 60.9, 40.0, 38.5, 34.6, 26.8.

### Example 20: (2E)-3-(pyrimidin-2-yl)-N-[2-(pyrimidin-2-yl)ethyl]prop-2-enamide

(E)-3-(pyrimidin-2-yl)acrylic acid (919 mg, 1.2 Eq, 6.12 mmol) was dissolved in DMF (25 mL). While stirring CDI (992 mg, 1.2 Eq, 6.12 mmol) was added and stirring was continued for 24 hours. Next day TEA (1.03 g, 1.42 mL, 2 Eq, 10.2 mmol) and 2-Pyrimidin-2-yl-ethylamine dihydrochloride (1.00 g, 1 Eq, 5.10 mmol) were added and mixture was stirred at 50° C. for three hours. The solvent was evaporated, and the residue was purified by flash column chromatography with eluent DCM/methanol. 0.3 g of the target compound was obtained.

¹H NMR (DMSO-d₆, 600 MHz) δ=8.80 (2H, d, J=4.8 Hz), 8.71 (2H, d, J=4.8 Hz), 8.52 (1H, br t, J=5.5 Hz), 7.40 (1H, t, J=4.8 Hz), 7.33 (1H, t, J=4.8 Hz), 7.27 (1H, d, J=15.1 Hz), 7.21 (1H, d, J=15.8 Hz), 3.62 (2H, q, J=6.9 Hz), 3.05 (2H, t, J=7.2 Hz). ¹³C NMR (DMSO-d₆, 151 MHz) δ=168.0, 164.1, 162.5, 157.7, 157.3, 137.5, 130.9, 120.6, 119.3, 38.5, 37.7.

### Example 21: 3,6-bis[(1H-imidazol-4-yl) methyl]piperazine-2,5-dione

A 30 mL vial was filled with methyl histidinate, 2HCl (5.00 g, 1 Eq, 20.7 mmol), TEA (4.18 g, 5.76 mL, 2 Eq, 41.3 mmol) and ethanol (7 mL). This vial was placed in the microwave and heated at 140° C. for three hours. Maximum pressure was 4 bar. Solids were filtered and washed with cold ethanol. After drying 0.4 g of yellow solid was obtained.

¹H NMR (D₂O, 600 MHz) δ=8.67 (1H, s), 7.33 (1H, s), 4.93-4.94 (1H, m), 4.24 (1H, t, J=4.5 Hz), 3.36 (1H, dd, J=15.1 Hz, J=4.8 Hz), 3.19 (1H, dd, J=15.8 Hz, J=4.8 H). ¹³C NMR (D₂O, 151 MHz) δ=171.1, 136.8, 130.0, 120.8, 56.5, 31.1.

### Example 22: Taste

The compound of example 1 ((2E)-3-(1H-imidazol-4-yl)-N-[2-(1H-imidazol-4-yl)ethyl]prop-2-enamide) have been tasted at different concentrations in water by a sensory panel. While no effect was noticed up to 40 ppm, there was a slight effect of mouth drying at 50 ppm or higher. This effect was noticeable up to 1000 ppm, while no taste as such was perceived, in particular no saltiness. At levels above 1000 ppm solubility issues occurred.

### Example 23: Salt Enhancement

Two aqueous solutions have been prepared:
A) 0.5% NaCl, and
B) 0.5% NaCl and 50 ppm of (2E)-3-(1H-imidazol-4-yl)-N-[2-(1H-imidazol-4-yl) ethyl]prop-2-enamide (compound of Example 1).

The solutions were tasted by a sensory panel. Solution A was describes as “salty”. Solution B was described as more salty with a mineralic note.

### Example 24: Salt Enhancement

The compound of example 1 ((2E)-3-(1H-imidazol-4-yl)-N-[2-(1H-imidazol-4-yl)ethyl]prop-2-enamide) have been tasted at different concentrations in a 0.3% NaCl solution by a sensory panel.

No salt enhancement was perceived below 1 ppm of the compound of example 1. At levels of 1 ppm or higher, there was recognized a salt enhancing effect, providing a more lingering, mineralic and fuller salt taste. Overall, the effect was perceived up to 200 ppm, with a preferred range of the compound between 50 and 70 ppm.

### Example 25: Umami Enhancement

Two aqueous solutions have been prepared:
A) 0.5% NaCl, 0.03% MSG, 0.007% Ribotides, and
B) 0.5% NaCl, 0.03% MSG, 0.007% Ribotides and 50 ppm of (2E)-3-(1H-imidazol-4-yl)-N-[2-(1H-imidazol-4-yl)ethyl]prop-2-enamide (compound of Example 1).

The solutions were tasted by a sensory panel. Solution A is a model solution for savoury taste, containing MSG (monosodium glutamate) and Ribotides (IMP/GMP, 50/50 mixture) as savoury taste enhancer. Solution B was described as having a strong boost of saltiness and umami in comparison to solution A.

### Example 26: Bitter Masking

Two aqueous solutions have been prepared:
A) 0.3% KCl, and
B) 0.3% KCl and 50 ppm of (2E)-3-(1H-imidazol-4-yl)-N-[2-(1H-imidazol-4-yl)ethyl]prop-2-enamide (compound of Example 1).

The solutions were tasted by a sensory panel. Solution B was described as less bitter and more salty and mineral in comparison to solution A.

### Example 27: Effect on Broth

A model broth base, comprising 0.3% of NaCl was compared with a sample further comprising 50 ppm of the compound of example 1.

The solutions were tasted by a sensory panel. The sample comprising the compound of example 1 was described as more mineral and salty in comparison to the model broth base. The sharp acidity of the broth base was reduced.

### Example 28: Effect on Cheese Sauce

The effect of the compound of example 1 on cheese sauce was explored. Therefore, a cheese sauce has been compared by a sensory panel with a sample of the sauce further comprising 50 ppm of the compound of example 1.

The sauce comprising the compound of example 1 was described as more mineral and salty, with a mineralic linger, and having a natural aged cheese character in comparison to the plain sauce.

### Example 29: Combination with Other Taste Modulating Compounds

Combinations of taste modulating compounds have been tasted in a 0.3% NaCl solution in water.
A) 50 ppm compound of example 1 and 50 ppm of N-lactoyl ethanolamine;
B) 50 ppm compound of example 1 and 0.04 ppm of N-oleoylmethionine; and
C) 50 ppm compound of example 1, 50 ppm N-lactoyl ethanolamine and 0.04 ppm N-oleoylmethionine.

The solutions were tasted by a sensory panel.

Sample A was described to show a clear combination of the two taste modulating compounds, providing a more salty and mineral taste with more body.

Sample B was perceived as even more salty with a good clear boost of the initial salt peak.

Sample C was having a boosted salt body with a salt peak being more round.

The combination of the three taste modulating compounds was preferred by the sensory panel.

### Example 30: Effect on Potato Chips

Potato chips with 1.5% salt have been tasted by a sensory panel with and without additional compound of example 1.

The potato chips further comprising the compound of example 1 at 70 ppm had a higher salty impact and a lingering effect in comparison to the potato chips without the compound of example 1.

### Example 31: Effect on Mayonnaise

Samples of commercially available mayonnaise with and without addition of the compound of example 1 have been compared by a sensory panel.

In comparison to the pure mayonnaise, the sample further comprising 70 ppm of the compound of example 1 was described as instant tingly salty with a mineral linger and enhanced acidity.

### Example 32: Effect on Corn Curls

Corn curls with cheese flavor with and without addition of the compound of example 1 have been compared by a sensory panel.

The taste of corn curls with added 70 ppm of the compound of example 1 was described as more salty, lingering and mineralic in comparison to the curls without the compound of example 1.

### Example 33: Effect on Vegan Cheese

Samples of vegan cheese comprising 0.2% cheese parmesan natural flavor have been compared with and without the compound of example 1 by a sensory panel.

By the addition of 70 ppm of the compound of example 1, the sample is described as more salty and mineral, and the overall taste is lifted.

### Example 34: Effect on Vegan Burger

Soy based vegan burgers with and without the compound of example 1 have been tasted by a sensory panel.

The burger with 70 ppm of the compound of example 1 is more salty, having a mineral linger, contributing well to the perception of a burger, when compared to the sample without the compound of example 1.

### Example 35: Effect on Processed Meat

Samples of processed meat with full salt flavor and sodium reduced flavor have been tasted by a sensory panel with and without the compound of example 1.

Sample 1 was comprising a full salt flavor base.

Sample 2 was comprising a full salt flavor base and 70 ppm of the compound of example 1.

Sample 3 was comprising a salt reduced (33.3%) flavor base and a saltiness flavor modulator.comprising KCl.

Sample 4 was comprising a salt reduced (33.3%) flavor base, a saltiness flavor modulator.comprising KCl, and 70 ppm of the compound of example 1.

Sample 2, containing 70 ppm of the compound of example 1 is perceived as more salty and mineral in comparison with sample 1 without the compound of the present invention. Compared to sample 1, sample 3 showed lower salty, increased astringency and dryness, with slight bitterness.

Compared to sample 1, sample 4 had improved salt-peak, increased salinity, prolonged saltiness, significantly reduced bitterness and astringency, and increased salivation.

### Example 36: Taste

The compounds of example 2-21 have been tasted in water by a sensory panel.

The solutions comprising 50 ppm of said compounds had no taste, respectively.

### Example 37: Salt Enhancement

Aqueous solutions comprising 0.5% NaCl and 50 ppm of the compounds of example 2-21, respectively, have been prepared and tasted by a sensory panel in comparison with an aqueous solution comprising 0.5% NaCl.

All solutions comprising the compounds of example 2-21 have been described as more salty in comparison the 0.5% NaCl aq. solution.

### Example 38: (2E)-3-(4H-imidazol-2-yl)-N-[2-(1H-imidazol-5-yl)ethyl]prop-2-enamide

1-hydroxypyrrolidine-2,5-dione (689 mg, 1.1 Eq, 5.99 mmol) was added to a yellow solution of (E)-3-(1H-imidazol-2-yl)acrylic acid hydrochloride (0.950 g, 1 Eq, 5.44 mmol) and TEA (1.10 g, 1.52 mL, 2 Eq, 10.9 mmol) in DMF (100 mL) and stirred for 5 minutes. Then dicyclohexylmethanediimine (1.23 g, 1.1 Eq, 5.99 mmol) was added and stirring was continued for 24 hours at rt. The next day, the formed dicyclohexylurea was filtered and the filtrate was placed in the refrigerator overnight. The precipitated dicyclohexylurea was filtered off. To the filtrate was added a solution of 2-(1H-imidazol-5-yl)ethan-1-amine (605 mg, 1 Eq, 5.44 mmol) in DMF (50 ml). The resulted mixture was stirred for 2 hrs at RT and 2 hrs at 55 C. After removal of solvent, the crude product was purified by silica gel column chromatography using eluent DCM/methanol. 0.8 g of the target (E)-N-(2-(1H-imidazol-5-yl)ethyl)-3-(1H-imidazol-2-yl) acrylamide was yielded as white solid. Purity is >95% by NMR analysis.

¹H NMR (DMSO-d₆, 600 MHz) δ=1.06-1.12 (1 H, m) 1.73-1.79 (1 H, m) 2.67 (2 H, t, J=7.23 Hz 2 H) 3.37-3.38 (2 H, m) 6.37 (1 H, d, J=15.84 Hz) 6.81 (1 H, s) 7.10 (1 H, dd, J=4.82, 3.44 Hz) 7.36 (1 H, d, J=3.44 Hz,) 7.54 (1 H, s) 7.56 (1 H, d, J=15.15 Hz) 7.59 (1 H, d, J=5.51 Hz) 8.16 (1 H, br t, J=5.85 Hz). ¹³C NMR (DMSO-d₆, 151 MHz,) δ=25.98, 38.49, 40.05, 116.57, 122.16, 127.27, 133.01, 134.28, 143.38, 164.89

### Example 39: (2E)-3-(1H-imidazol-4-yl)-N-[2-(1H-imidazol-2-yl)ethyl]prop-2-enamide

(E)-N-(2-(1H-imidazol-2-yl)ethyl)-3-(1H-imidazol-4-yl)acrylamide was synthesized using the same procedure as described in example 38. (E)-3-(1H-imidazol-4-yl)acrylic acid (825 mg, 1.1 Eq, 5.98 mmol) was coupled with 2-(1H-imidazol-2-yl)ethan-1-amine, 2HCl (1.00 g, 1 Eq, 5.43 mmol) to obtain 0.8 g of the target compound as pale yellow solid. Purity is >90% by NMR analysis.

¹H NMR (DMSO-d₆, 600 MHz) δ=8.36 (1H, br t, J=5.9 Hz), 7.75 (1H, s), 7.49 (2H, s, H-11), 7.39 (1H, s), 7.29 (1H, d, J=15.8 Hz), 6.46 (1H, d, J=15.8 Hz), 3.56 (2H, q, J=6.2 Hz), 3.22-3.28 (1H, m), 3.08 (2H, t, J=6.5 Hz). ¹³C NMR (DMSO-d₆, 151 MHz) δ, 145.3, 137.1, 135.3 (C-16), 130.9 (C-4), 121.3 (C-17), 119.1 (C-11, 12), 118.0 (C-3), 36.9 (C-6), 26.3 (C-7)

### Example 40: N-(2-hydroxyethyl)-3-(1H-imidazol-4-yl)propanamide

(E)-N-(2-hydroxyethyl)-3-(1H-imidazol-4-yl)acrylamide (0.5 g, 2.76 mmol) was dissolved in methanol (40 ml) to give a pale yellow solution. The solution was purged with nitrogen for 5 minutes, then Pd-C 10% (60 mg, 0.564 mmol) was added to the solution. The reaction mixture was stirred at rt under 1 atm hydrogen until no more hydrogen was consumed. The catalyst was filtered and the filtrate was evaporated. The residual solid was washed with ether and dried in vacuum oven at 50° C. 0.5 g of the target N-(2-hydroxyethyl)-3-(1H-imidazol-4-yl)propanamide was obtained as white solid. Purity is >95% by NMR analysis.

¹H NMR (DMSO-d₆, 600 MHz) δ=8.78 (1H, s), 7.27 (1H, s), 3.35 (2H, t, J=5.9 Hz), 3.08 (2H, t, J=6.2 Hz), 2.84 (2H, t, J=7.6 Hz), 2.46 (2H, t, J=7.6 Hz). ¹³C NMR (DMSO-d₆, 151 MHz) δ=172.0, 133.4, 133.0, 115.8, 60.0, 41.8, 34.0, 20.4

### Example 41: (2E)-3-(1H-imidazol-4-yl)-N-[2-(1H-pyrrol-2-yl)ethyl]prop-2-enamide

(E)-3-(1H-imidazol-4-yl) acrylic acid (1.00 g, 1 Eq, 7.24 mmol) was dissolved in DMF (25 mL). CDI (Carbonyldiimidazole) (1.41 g, 1.2 Eq, 8.69 mmol) was added while stirring at RT and stirring was continued for 24 hours at RT. 2-(1H-pyrrol-2-yl) ethan-1-amine (798 mg, 1 Eq, 7.24 mmol) was added, and the reaction mixture was stirred at 50° C. for three hours. Solvent was evaporated and residue was purified by flash column chromatography yielding 0.3 g of a light brown solid. Purity is >95% by NMR analysis.

¹H NMR (D₂O, 600 MHz) δ=8.60 (1H, s), 7.55 (1H, m), 7.20 (1H, d, J=15.8 Hz), 6.44 (1H, d, J=15.8 Hz), 3.39 (2H, t, J=6.5 Hz), 2.71 (2H, t, J=6.9 Hz), other protons were exchanged by deuterium due to solvent (see multiplets in ¹³C NMR). ¹³C NMR (D₂O, 151 MHz) δ=166.9, 134.9, 133.3, 129.5, 129.0, 124.7, 123.5, 119.6, 118.9, 117.4 (1C, t, J=25.43 Hz), 107.2 (1C, t, J=24.98 Hz) 104.9 (1C, t, J=25.88 Hz) 39.7, 26.5.

### Example 42:3-[2-amino-3-(1H-imidazol-4-yl)propanamido]propanoic acid

The target was synthesized by saponification of methyl 3-(2-amino-3-(1H-imidazol-4-yl)propanamido)propanoate (0.91 g; 3.79 mmol) with sodium hydroxide (0.30 g; 7.58 mmol) in water (100 ml). After acidification with dilute hydrochloric acid, the precipitated solid was filtered, washed with methanol and dried in vacuum oven. 0.84 g of the target compound was obtained as white solid. Purity is >95% by NMR analysis.

¹H NMR (D₂O, 600 MHz) δ=8.56 (1H, s), 7.38 (1H, s), 4.22 (1H, t, J=6.5 Hz), 3.46-3.55 (1H, m), 3.31-3.37 (1H, m), 3.29-3.40 (3H, m), 2.36 (2H, td, J=6.5 Hz, J=2.1 Hz), 1.41 (1H, s), 1.37 (1H, s). ¹³C NMR (D₂O, 151 MHz) δ=182.7, 170.9, 137.6, 129.6, 121.1, 55.3, 51.8, 39.6, 39.0, 29.5.

### Example 43: (2Z)-3-(1H-imidazol-4-yl)-N-[2-(1H-imidazol-4-yl)ethyl]prop-2-enamide

An amount of 2.00 g of trans-urocanic acid was solved in 1000 ml of demineralized water. The pH was adjusted to pH 9.00 with using a 1 M NaOH solution. The obtained mixture was placed in an UV-reactor and cooled to 0° C. A constant slow nitrogen bubbling flow was passed through this solution. The isomerization started by UV illumination with using a UV lamp N1 from Heraeus Noblelight during 7 hrs at 0° C. The solution was then brought to pH 3.8 with addition of a 1 M HCl solution. The obtained solution was then freeze dried during 120 hrs at 0.5 mbar. An amount of 2.00 g of an off white coloured mixture was obtained. NMR of the mixture showed a cis: trans ratio of 0.85:1.

2.00 g of the cis-trans-urocanic acid mixture with a ratio of 0.85:1 was solved in 90 ml of anhydrous DMF. An amount of 1.83 g of N-hydroxysuccinimide was added and the mixture was stirred for 10 min. A solution of 3.29 g of DCC in 30 ml of anhydrous DMF was added dropwise to the mixture during 10 min. Stirring continued then for another 24 hrs at r.t. The obtained mixture was filtered and the residue was washed with 2*5 ml anhydrous DMF. The filtrated was stored overnight at −18° C. under a N2 atmosphere and filtered again. The residue was washed with 2*5 ml anhydrous DMF. To the obtained filtrate was added dropwise while stirring a solution of 2.43 g of sodium bicarbonate and 2.67 g of histamine dihydrochloride in 22 ml of water. The reaction mixture was then warmed to 45° C. and stirring continued for 5 hrs. The reaction mixture was allowed to stand overnight at r.t. The obtained mixture was evaporated till dryness with using a rotavapor. The obtained residue was stirred with 25 ml of methanol for 1 hr. A white precipitate was formed and filtered. The filtrate was evaporated again till dryness. To the obtained residue was added an amount of 25 ml of absolute ethanol. The mixture was again stirred for 1 hr and subsequently filtered. The ethanol was removed by evaporation. Purification was then done with using flash chromatography with DCM: MeOH as eluents. An amount of 0.25 g of (Z)-N-(2-(1H-imidazol-4-yl)ethyl)-3-(1H-imidazol-4-yl)acrylamide was obtained.

¹H NMR (DMSO-d₆, 600 MHz) δ=7.77 (1H, s), 7.56 (1H, s), 7.27-7.36 (1H, m), 6.82 (1H, s, 6.70 (1H, d, J=12.4 Hz), 5.68 (1H, d, J=12.4 Hz3), 3.28-3.46 (2H, m), 2.71 (2H, t, J=7.2 Hz). ¹³C NMR (DMSO-d₆, 151 MHz) δ=167.1, 137.7, 135.2, 127.5, 116.6, 39.6, 27.3.

### Example 44: (2E)-N-[2-(1H-imidazol-4-yl)ethyl]-3-phenylprop-2-enamide

A solution of cinnamoyl chloride (7.2 g, 43.2 mmol) in Dichloromethane (50.0 ml) was added dropwise to a solution of 2-(1H-imidazol-4-yl)ethan-1-amine (5.2 g, 46.8 mmol) and TEA (13.77 ml, 99 mmol) in ethanol (50 ml) cooled with an ice bath. After addition, the cooling bath was removed and stirring was continued for 1 hour, then the reaction mixture allowed to stand at room temperature overnight. Then DCM and ethanol were removed by evaporation under reduced pressure. The crude product was purified by washing with DCM, ether and pentane and recrystallization from ethanol. 8.0 g of the target N-(2-(1H-imidazol-4-yl)ethyl)cinnamamide was yielded as white powder. Purity is >95% by NMR analysis.

¹H NMR (DMSO-d₆, 600 MHz) δ=8.21 (1H, t, J=5.5 Hz), 7.53-7.58 (3H, m), 7.42 (1H, d, J=15.8), 7.40 (2H, t, J=7.5 Hz), 7.6 (1H, t, J=7.5 Hz), 6.82 (1H, s), 6.64 (1H, d, J=15.8 Hz), 3.41 (2H, m) 2.69 (2H, t, J=7.4 Hz). ¹³C NMR (DMSO-d₆, 151 MHz) δ=164.9, 138.5, 135.0, 134.7, 129.4, 129.0, 127.5, 122.3, 38.9, 27.1.

### Example 45: (2E)-N-benzyl-3-(1H-imidazol-4-yl) prop-2-enamide

(E)-3-(1H-imidazol-4-yl)acrylic acid (2.00 g, 1 Eq, 14.5 mmol) was dissolved in DMF (75 mL). 1-hydroxypyrrolidine-2,5-dione (1.83 g, 1.1 Eq, 15.9 mmol) was added.

Dicyclohexylmethanediimine (3.29 g, 1.1 Eq, 15.9 mmol) was added, and stirring was continued for 24 hours at RT. The formed solids (dicyclohexylurea) were filtered and to the filtrate phenylmethanamine (1.71 g, 1.1 Eq, 15.9 mmol) was added. The reaction mixture was stirred at 50° C. for three hours. Solvent was evaporated, and residue was taken up in methanol. Silica (15 g) was added and solvent was evaporated. Product was purified by flash column chromatography yielding in 1.8 g of solid sample material. Spectra are in accordance with the target structure in a purity of >95%.

¹H NMR (METHANOL-d₄, 600 MHz) δ=7.75 (1H, s), 7.48 (1H, d, J=15.1 Hz), 7.32-7.36 (2H, m), 7.31 (2H, br s), 7.22-7.29 (1H, m), 6.54 (1H, br d, J=15.1 Hz), 4.48 (2H, s). ¹³C NMR (METHANOL-d₄, 151 MHz) δ=169.1 (C-1), 140.1 (C-1), 138.6 (C-2), 129.7 (C-3, 5), 128.8 (C-2, 6), 128.4 (C-4), 119.3 (C-2 (E)), 44.5.

### Example 46: (2E)-3-(1H-imidazol-4-yl)-N-[(pyrimidin-5-yl)methyl]prop-2-enamide

(E)-3-(1H-imidazol-4-yl)acrylic acid (1.00 g, 1 Eq, 7.24 mmol) was dissolved in DMF (25 mL). CDl (1.41 g, 1.2 Eq, 8.69 mmol) was added while stirring at RT, and stirring was continued for 24 hours at RT. 5-Aminomethylpyrimidine (790 mg, 1.00 Eq, 7.24 mmol) was added, and reaction mixture was stirred at 50° C. for three hours. Solvent was evaporated, and residue was purified by flash column chromatography yielding in 0.1 g of a white solid. Structure is confirmed by NMR in high purity >95%.

¹H NMR (D₂O, 600 MHz) δ=9.07 (1H, s), 8.77-8.81 (2H, m), 8.76-8.79 (1H, m), 7.75 (1H, s), 7.46 (1H, d, J=16.5 Hz), 6.72 (1H, d, J=15.8 Hz), 4.60 (2H, s). ¹³C NMR (D₂O, 151 MHz) δ=170.5, 159.3, 159.2, 138.1, 135.3, 132.0, 128.7, 125.9, 122.9, 41.7.

### Example 47: (2E)-3-(1H-imidazol-4-yl)-N-[(pyrazin-2-yl)methyl]prop-2-enamide

(E)-3-(1H-imidazol-4-yl)acrylic acid (5.00 g, 1 Eq, 36.2 mmol) was dissolved in DMF (125 mL). N,N′-Carbonyldiimidazole (7.04 g, 1.2 Eq, 43.4 mmol) was added while stirring at RT, and stirring was continued for 24 hours at RT. Pyrazin-2-ylmethanamine (3.95 g, 1 Eq, 36.2 mmol) was added, and reaction mixture was stirred at 50° C. for three hours. Solvent was evaporated, and residue was purified by flash column chromatography yielding 3 g of product in a purity of >95%.

¹H NMR (D₂O, 600 MHz) δ=8.64 (2H, s,), 8.59 (1H, s), 8.50 (1H, d, J=2.7 Hz), 7.61 (1H, s,), 7.30 (1H, d, J=15.9 Hz), 6.61 (1H, d, J=15.8 Hz), 4.61 (2H, s). ¹³C NMR (D₂O, 151 MHz) δ170.6, 157.3, 148.1, 143.8, 143.7, 138.1, 131.9, 128.7, 125.8, 123.0, 45.6.

### Example 48: (2E)-3-(furan-2-yl)-N-[2-(1H-imidazol-4-yl)ethyl]prop-2-enamide

(E)-N-(2-(1H-imidazol-4-yl)ethyl)-3-(furan-2-yl)acrylamide was synthesized using the same procedure as described for N-(2-(1H-imidazol-4-yl)ethyl)cinnamamide (example 44). 2-(1H-imidazol-4-yl)ethan-1-amine (2.00 g, 1 Eq, 18.0 mmol) was reacted with (E)-3-(furan-2-yl)acryloyl chloride (3.10 g, 1.10 Eq, 19.8 mmol) to yield 1.6 g of the target (E)-N-(2-(1H-imidazol-4-yl)ethyl)-3-(furan-2-yl)acrylamide as beige powder. Purity is >95% by NMR analysis.

¹H NMR (DMSO-d₆, 600 MHz) δ=8.94 (1H, s), 8.47 (1H, br t, J=5.5 Hz), 7.76 (1H, s), 7.41 (1H, s), 7.21 (1H, d, J=15.8 Hz), 6.76 (1H, d, J=2.8 Hz), 6.57 (1H, br s), 6.41 (1H, d, J=15.8 Hz,), 3.47 (2H, q, J=6.2 Hz), 2.83 (2H, br t, J=6.5 Hz) ¹³C NMR (DMSO-d₆, 151 MHz) δ=165.0, 150.9, 144.8, 133.6, 131.2, 126.1, 119.3, 116.2, 113.9, 112.4, 55.0, 37.8, 24.6.

### Example 49: (2E)-N-[2-(1H-imidazol-4-yl)ethyl]-3-(thiophen-2-yl)prop-2-enamide

2-(1H-imidazol-4-yl)ethan-1-amine (1.00 g, 1 Eq, 9.00 mmol) was dissolved in methanol (20 mL) and diluted with DCM (50 mL). Triethylamine (2.73 g, 3 Eq, 27.0 mmol) was added, and then a solution of (E)-3-(thiophen-2-yl) acryloyl chloride (1.86 g, 1.2 Eq, 10.8 mmol) in DCM (50 mL) was added dropwise at rt. After 2 hrs stirring at rt, the solution was evaporated. The residual solid was washed with DCM then ethyl acetate and ether. The solid was taken in THF (150 ml) and agitated at 50 C for 15 minutes. The insoluble solid was filtered. The THF solution was cooled to rt and then diluted with ether until precipitation occurred. The white precipitate was filtered, washed with ether and then dried in vacuum oven at 40 C. 0.7 g of (E)-N-(2-(1H-imidazol-4-yl)ethyl)-3-(thiophen-2-yl)acrylamide was yielded as white powder. Purity is >95% by NMR analysis.

¹H NMR (DMSO-d₆, 600 MHz) δ=8.16 (1H, br t, J=5.9 Hz), 7.59 (1H, d, J=5.5 Hz), 7.56 (1H, d, J=15.1 Hz), 7.54 (1H, s), 7.36 (1H, d, J_{17,20}=3.4 H), 7.10 (1H, dd, J=4.8 Hz, J=3.4 Hz), 6.81 (1H, s), 6.37 (1H, d, J=15.8 Hz), 3.37-3.38 (2H, m), 2.67 (2H, t, J=7.2 Hz). ¹³C NMR (DMSO-d₆, 151 MHz) δ=164.6, 139.9, 134.7, 131.6, 130.6, 128.3, 127.8, 121.0, 38.9, 25.1.

### Example 50: (2E)-N-[2-(1H-imidazol-4-yl)ethyl]-3-(thiophen-3-yl)prop-2-enamide

Histamine (1.1 g, 1 Eq, 9.9 mmol) was dissolved in methanol (10 mL) and diluted with DCM (50 mL). TEA (3.0 g, 4.1 mL, 3 Eq, 30 mmol) was added, and then a solution of (E)-3-(thiophen-3-yl)acryloyl chloride (2.0 g, 1.2 Eq, 12 mmol) in DCM (50 mL) was added dropwise at rt. After 2 hrs stirring at rt the solution was evaporated. The remaining residual solid was transferred to silica gel column chromatography and then eluted with DCM/methanol to yield 1.1 g of the target (E)-N-(2-(1H-imidazol-4-yl)ethyl)-3-(thiophen-3-yl)acrylamide as off white powder. Purity is >95% by NMR.

¹H NMR (DIMETHYLFORMAMIDE-d₇, 600 MHz) δ=7.82 (1H, d, J=2.1 Hz), 7.63-7.66 (1H, m), 7.61-7.66 (1H, m), 7.54 (1H, d, J=15.1 Hz), 7.41 (1H, d, J=4.8 Hz), 6.92 (1H, s), 6.60 (1H, d, J=15.8 Hz), 3.53-3.58 (2H, m), 2.81 (2H, t, J=7.6 Hz). ¹³C NMR (DIMETHYLFORMAMIDE-d₇, 151 MHz) δ=165.8, 138.8, 135.2, 133.0, 127.6, 127.3, 125.5, 122.5, 39.7, 27.7.

### Example 51: 3-(1H-imidazol-4-yl)-2-{[(2E)-3-phenylprop-2-enoyl]amino}propanoic acid

Synthesis: L-histidine (3.00 g, 1 Eq, 19.3 mmol) was dissolved in aqueous solution of sodium hydroxide (1.8 g, 2.3 Eq, 44.5 mmol) in water (50 mL) and diluted with THF (50 mL). Then a solution of cinnamoyl chloride (4.19 g, 1.3 Eq, 25.1 mmol) in THF (50 mL) was added dropwise at rt. The reaction mixture was stirred at rt for 3 hours, then neutralized with 1M HCl and then evaporated under reduced pressure at 30 C. The remaining solid was purified by silica gel column chromatography using DCM/methanol. 0.5 g of the target compound cinnamoyl-L-histidine was yielded as white solid. Purity is >95% by NMR.

¹H NMR (METHANOL-d₄, 600 MHz) δ=7.95 (1H, s), 7.55 (2H, br d, J=6.2 Hz), 7.48 (1H, d, J=15.8 Hz,), 7.35-7.39 (2H, m), 7.33-7.39 (1H, m), 7.00 (1H, s), 6.70 (1H, d, J=15.8 Hz), 4.66 (1H, dd, J=6.9 Hz, J_{α,β<″>}=4.8 Hz), 3.25 (1H, dd, J_{β<″>,β<′>}=15.1 Hz, J_{β<″>,α}=4.8 Hz), 3.10 (1H, dd, J_{β<′>,β<″>}=15.1 Hz, J=7.6 Hz. ¹³C NMR (METHANOL-d₄, 151 MHz) δ=177.3, 168.0, 141.8, 136.5, 135.4, 133.1, 130.9, 130.1, 129.0, 122.3, 119.9, 55.9, 30.4.

### Example 52: 3-(1H-imidazol-4-yl)-2-{[(2E)-2-methylbut-2-enoyl]amino}propanoic acid

(E)-(2-methylbut-2-enoyl)-L-histidine was synthesized using the same procedure as described for cinnamoyl-L-histidine (example 51). L-histidine (2.50 g. 1 Eq. 16.1 mmol) was reacted with (E)-2-methylbut-2-enoyl chloride (2.00 g, 1.05 Eq, 16.9 mmol). 0.8 g of the target compound was yielded as white solid. Purity is >98% by NMR.

¹H NMR (DMSO-d₆, 600 MHz) δ=7.95 (1H, d, J=7.6 Hz), 7.60 (1H, s), 6.81 (1H, s), 6.34 (1H, qd, J=6.9 Hz, J=1.4 Hz), 4.38 (1H, d, J=6.9 Hz), 2.96 (2H, d, J=6.2 Hz, H-9), 1.72 (3H, s), 1.69 (3H, d, J=7.6 Hz). ¹³C NMR (DMSO-d₆, 151 MHz) δ=173.4, 168.0, 134.7, 133.7, 131.5, 129.9, 116.8, 52.8, 28.4, 13.7, 12.3.

### Example 53: N-[2-(1H-imidazol-4-yl)ethyl]-2-methylbutanamide

N-(2-(1H-imidazol-4-yl)ethyl)-2-methylbutanamide was synthesized using the same procedure as described for (E)-N-(2-(1H-imidazol-4-yl) ethyl)-3-(thiophen-3-yl)acrylamide (example 50). Histamine (1.5 g, 1 Eq, 13 mmol) was reacted with 2-methylbutanoyl chloride (1.6 g, 1 Eq, 13 mmol). 0.4 g of the target N-(2-(1H-imidazol-4-yl) ethyl)-2-methylbutanamide was yielded as pale yellow solid. Purity is >95% by NMR.

¹H NMR (DMSO-d₆, 600 MHz) δ=7.80-7.92 (1H, m), 7.57 (1H, s), 6.79 (1H, s), 3.20-3.32 (2H m), 2.62 (2H, t, J=7.2 Hz), 2.07-2.13 (1H, m), 1.42-1.50 (1H, m), 1.22-1.31 (1H, m), 0.95 (3H, d, J=6.9 Hz), 0.76 (3H, t, J=7.6 Hz). ¹³C NMR (DMSO-d₆, 151 MHz) δ=175.4, 134.6, 134.2), 116.9, 41.4, 38.5, 27.0, 26.9, 17.7, 11.8.

### Example 54: (2E)-N-[2-(1H-imidazol-4-yl)ethyl]-3.7-dimethylocta-2,6-dienamide

(E)-N-(2-(1H-imidazol-4-yl)ethyl)-3,7-dimethylocta-2,6-dienamide was synthesized using the same procedure as described for (E)-N-(2-(1H-imidazol-4-yl)ethyl)-3-(thiophen-3-yl)acrylamide (example 50). Histamine (1.00 g, 1 Eq, 9.00 mmol) was reacted with (E)-3,7-dimethylocta-2,6-dienoyl chloride (2.50 g, 1.49 Eq, 13.4 mmol). 0.5 g of the target N-(2-(1H-imidazol-4-yl)ethyl)-2-methylbutanamide was yielded as white solid. Purity is >95% by NMR.

¹H NMR (DMSO-d₆, 600 MHz) δ=7.82 (1H, br t, J=5.2 Hz), 7.52 (1H, s), 6.77 (1H, br s), 5.62 (1H, br s), 5.05-5.11 (1H, m), 3.25-3.30 (3H, m), 2.62 (2H, br t, J=7.2 Hz), 2.06-2.12 (1H, m), 2.05-2.09 (1H, m), 2.01-2.06 (1H, m), 2.01-2.06 (3H, m), 1.63-1.66 (3H, m), 1.53-1.61 (4H, m). ¹³C NMR (DMSO-d₆, 151 MHz) δ=166.0, 151.4, 134.6, 131.4, 123.5, 118.8, 40.2, 38.5, 25.7, 25.5, 17.6.

### Example 55: 2-{[(2E)-3-(furan-2-yl)prop-2-enoyl]amino}-3-(1H-imidazol-4-yl)propanoic Acid

L-histidine (3.00 g, 1 Eq, 19.3 mmol) was dissolved in aqueous solution of sodium bicarbonate (4.06 g, 2.5 Eq, 48.3 mmol) in water (50 mL) and diluted with THF (30 mL). Then a solution of (E)-3-(furan-2-yl) acryloyl chloride (3.94 g, 1.3 Eq, 25.1 mmol) in THF (30 mL) was added dropwise at rt. The reaction mixture was stirred at rt overnight, then neutralized with 1M HCl. The reaction mixture was extracted with ethyl acetate (2× 150 ml) to remove the unreacted (E)-3-(furan-2-yl) acrylic acid. The water layer was evaporated under reduced pressure at 30 C. The remaining solid was suspended in methanol (200 ml), agitated for 15 minutes and then filtered. The filtrate was evaporated. The remaining solid was suspended in methanol (200 ml) again, agitated and then filtered. The filtrate was evaporated. The remaining residual solid was transferred to silica gel column chromatography and then eluted with DCM/methanol to yield 1.3 g of the target compound as off white solid. Purity is >95% by NMR.

¹H NMR (D₂O, 600 MHz) δ=7.73 (1H, s), 7.58 (1H, d, J=1.4 Hz), 7.25 (1H, d, J=15.1 Hz), 6.96 (1H, s), 6.72 (1H, d, J_{17,18}=3.4 Hz), 6.55 (1H, dd, J_{18,17}=3.4 Hz, J=2.1 Hz), 6.44 (1H, d, J=15.8 Hz), 4.57 (1H, dd, J_{12,11}=9.0 Hz, J_{12,11}=4.8 Hz), 3.19 (1H, dd, J_{11,11}=14.8 Hz, J_{11,12}=4.5 Hz), 3.02 (1H, dd, J_{11,11}=15.1 Hz, J_{11,12}=9.0 Hz). ¹³C NMR (D₂O, 151 MHz) δ=180.9, 170.8, 153.6, 147.9, 138.4, 135.8, 131.1, 120.4, 120.0, 117.7, 115.3, 58.2, 32.1.

### Example 56 :3-(1H-imidazol-5-yl)-2-{[(2E)-3-(1H-imidazol-4-yl)prop-2-enoyl]amino}propanoic Acid

The reaction was carried out under dry conditions with a slow nitrogen flow. The activated ester of urocanic acid with N-hydroxysuccinimide was first prepared by solving (E)-3-(1H-imidazol-4-yl)acrylic acid (1.727 g, 1 Eq, 12.50 mmol) in 80 ml of anhydrous DMF while stirring. To this solution was added 1-hydroxypyrrolidine-2,5-dione (1.582 g, 1.1 Eq, 13.75 mmol) and stirring continued for about 10 min at r.t. To the mixture was then added dropwise during 10 min, a solution of dicyclohexylmethanediimine (2.837 g, 1.1 Eq, 13.75 mmol) in 25 ml of anhydrous DMF. The obtained mixture was stirred for 24 hrs at r.t. The reaction mixture was then filtered to remove the formed dicyclohexylurea. The residue was washed with 2*5 ml of anhydrous DMF and the obtained clear filtrate was stored under a nitrogen atmosphere at −18° C. during the night and filtered again. The residue was washed with 2*5 ml of anhydrous DMF. Obtained is a solution from the activated ester of 12.5 mmol of urocanic acid in about 100 ml of anhydrous DMF.A 250 ml reaction flask was charged with 12.5 mmol of the obtained activated ester solution of urocanicacid in DMF. A solution of L-histidine dihydrochloride (2.851 g, 1 Eq, 12.50 mmol) and sodium hydrogencarbonate (2.993 g, 2.85 Eq, 35.63 mmol) in 20 ml of water was added dropwise during 15 min at r.t. During the addition, the pH of the reaction mixture was kept at pH 9 by using a 1 M NaOH solution. After addition, the temperature was kept at 40° C. during 4 hrs. The reaction mixture was cooled to r.t. Then the pH was adjusted to pH 2.5 with careful addition of 37% HCl. An amount of 100 ml of water was added to the mixture and washed with 3*20 ml of ethyl acetate. The water phase was then evaporated till dryness with using a rotavapor. To the obtained residue was added an amount of 50 ml of absolute ethanol and stirred over night at r.t. The mixture was filtered. The obtained filtrate was evaporated and the obtained residue was washed with 30 ml of acetonitrile, filtered and evaporated till dryness. LC-MS analyses of the obtained residue confirmed the desired mol weight present for the product. Purification was done with using RPC18-prep HPLC. Isolated was 100 mg of the desired product.

¹H NMR (600 MHz, D₂O) δ=8.70 (1 H, s), 8.60 (1 H, d, J=1.4 Hz), 7.71 (1 H, br s), 7.38 (1 H, d, J=15.8 Hz), 7.28 (1 H, br s), 6.67 (1 H, d, J=15.8 Hz), 4.66 (1 H, dd, J=8.3, 4.8 Hz), 3.32-3.36 (1 H, m), 3.17 (1 H, dd, J=15.5, 8.6 Hz). ¹³C NMR (151 MHz, D₂O) δ=178.9, 169.4, 138.1, 136.0, 132.4, 132.3, 128.9, 125.3, 122.9, 119.6, 57.2, 30.2.

### Example 57: N-[2-(1H-imidazol-5-yl)ethyl]-1,3-benzothiazole-6-carboxamide

A suspension of 2-(1H-imidazol-4-yl)ethan-1-amine, 2HCl (1.473 g, 8.00 mmol) in pyridine (25 ml) was stirred in an ice/water bath. Benzo[d]thiazole-6-carbonyl chloride (1.976 g, 10 mmol) was added. The reaction mixture was stirred in an ice/water bath for 30 minutes and at RT for 24 hours. Pyridine was evaporated, residue was taken up in water, and this suspension was brought to pH=11 by adding a sodiumhydroxide solution. The waterlayer was extracted with ethylacetate. The combined organic layers were dried and evaporated. The residue, which was a small amount, was mainly desired product but not pure. The waterlayer was also evaporated and the residue was taken up in methanol. Remaining solids (NaCl) were filtered and filtrate was evaporated. The residue was also mostly desired product. Purification was done by flash column chromatography with a part of the crude yielding 0.2 g of a white solid in a pruity of >95% according to NMR.

¹H NMR (600 MHz, DMSO-d₆) δ=9.53 (1 H, s), 8.74 (1 H, t, J=5.2 Hz), 8.64 (1 H, s), 8.15 (1 H, d, J=9.0 Hz), 7.99 (1 H, d, J=8.5 Hz), 7.55 (1 H, s), 6.84 (1 H, s), 3.52 (2 H, q, J=6.9 Hz), 2.78 (2 H, t, J=7.6 Hz). ¹³C NMR (151 MHz, DMSO-d₆) δ=165.6, 158.8, 154.6, 134.7, 133.7, 131.9, 125.3, 122.7, 122.0, 40.0, 33.4, 27.0.

### Example 58: (2E)-3-(1H-imidazol-4-yl)-N-(2-phenylethyl) prop-2-enamide

(E)-3-(1H-imidazol-4-yl)acrylic acid (15 g, 109 mmol) was dissolved in Dioxane (250 ml). To this emulsion was added 1-hydroxypyrrolidine-2,5-dione (13.75 g, 119 mmol) and DCC (24.65 g, 119 mmol). The mixture was stirred for 24 hours at room temperature. Solids were filtered. A part (⅓) of the filtrate was used in the next reaction step. To 92 g dioxane solution of the filtrate was added 2-phenylethan-1-amine (4.40 g, 36.3 mmol). This solution was stirred at 50° C. for three hours. Dioxane was evaporated and residue was taken up in DCM. Solids were removed by filtration and the filtrate was washed with a sodiumbicarbonate solution. Organic layer was separated, dried and evaporated. The residue was purified by flash column chromatography yielding 1 g of a light yellow solid. The solid was further purified by preparative HPLC yielding 80 mg of product in a purity of >98% according to NMR.

¹H NMR (600 MHz, DMSO-d₆) δ=12.22 (1 H, br s), 8.10 (1 H, br t, J=5.5 Hz), 7.69 (1 H, s), 7.39 (1 H, s), 7.26-7.31 (4 H, m), 7.18-7.24 (4 H, m), 6.51 (1 H, d, J=15.1 Hz), 3.37-3.40 (2 H, m), 2.75 (2 H, t, J=7.2 Hz). ¹³C NMR (151 MHz, DMSO-d₆) δ=165.7, 139.6, 137.3, 136.8, 131.6, 128.7, 128.3, 126.1, 118.8, 118.4, 40.3, 35.3.

### Example 59: Salt Enhancement

Aqueous solutions comprising 0.5% NaCl and 50 ppm of the compounds of example 38-58, respectively, have been prepared and tasted by a sensory panel in comparison with an aqueous solution comprising 0.5% NaCl.

All solutions comprising the compound of example 38-58 have been described as more salty in comparison the 0.5% NaCl aq. solution. In addition, some examples were described as more metallic, rich, umami, mineralic, with a cleaner salty taste and slight sourness.

### Example 60: Umami Enhancement

Two aqueous solutions have been prepared:
A) 0.5% NaCl, 0.03% MSG, 0.007% Ribotides, and
B) 0.5% NaCl, 0.03% MSG, 0.007% Ribotides and 50 ppm of the compound of example 38-58, respectively.

The solutions were tasted by a sensory panel. Solution A is a model solution for savoury taste, containing MSG (monosodium glutamate) and Ribotides (IMP/GMP, 50/50 mixture) as savoury taste enhancer. Solution B comprising the compound of example 38-58, respectively, was described as having a strong boost of saltiness and umami in comparison to solution A.

In addition, some examples were described as more rich, mineralic, with slight sourness.

1. A method of modulating a flavor, the method comprising adding one or more compounds of formula (I) 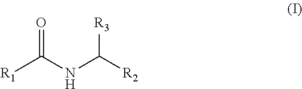
and edible salts thereof, wherein
R₁ is selected from the group consisting of 2-(1H-4-imidazolyl)-ethenyl, 1H-5-indolyl, 2-(1H-5-imidazolyl)-ethenyl, 1-amino-2-(1H-4-imidazolyl)-ethyl, (1,3-thiazol-2-yl)-ethenyl, 2,3-dihydro-1H-indol-2-yl, 2-(pyrimidin-2-yl)ethenyl, heptadecanyl, 1-heptadec-8-enyl, heptadeca-8,11-dienyl, heptadeca-8,11, 14-trienyl, 2-(4H-imidazol-2-yl)-ethenyl, 2-(4H-imidazol-2-yl)-ethyl, 2-phenyl-ethenyl, 2-(furan-2-yl)-ethenyl, 2-(thiophen-2-yl)-ethenyl, 2-(thiophen-3-yl)-ethenyl, 2-but-2-enoyl, 2-butyl, 2,6-dimethylhepta-1,5-dienyl, 1,3-benzothiazol-6-yl;
R₂ is selected from the group consisting of (1H-imidazol-4-yl)-methyl, (1H-3-indol-3-yl)-methyl, 4-hydroxybenzyl, methylsulfanylethyl, hydroxymethyl, CH₂-COOH, (pyridin-4-yl)methyl, (pyridin-2-yl)methyl, 1-(2,6-dimethylhepta-1,5-dienyl), 2-(pyrimidin-2-yl)methyl, (1H-imidazol-5-yl)-methyl, (1H-imidazol-2-yl)-methyl, (1H-pyrrol-2-yl)-methyl, phenyl, pyrimidin-5-yl, pyrazin-2-yl;
R₃ is selected from the group consisting of H, COOH,
or a compound selected from the group consisting of 3-(1H-imidazol-4-yl) prop-2-enamide, methyl 2,3-dihydro-1H-indole-2-carboxylate, and 3,6-bis[(1H-imidazol-4-yl)methyl]piperazine-2,5-dione,
to a flavor composition or a consumer product.

2. The method according to claim 1, wherein the compound is selected from the group consisting of 3-(1H-imidazol-4-yl)-N-[2-(1H-imidazol-4-yl)ethyl]prop-2-enamide, N-[2-(1H-imidazol-4-yl)ethyl]-1H-indole-5-carboxamide, Histidyltyrosine, N-[3-(1H-imidazol-5-yl)prop-2-enoyl]-tryptophan, Histidylhistidine, N-[3-(methylsulfanyl)propyl]histidinamide, N-(2-hydroxyethyl)histidinamide, Histidyl-β-alanine, N-[octadeca-9,12-dienoyl]histidine, N-[2-(1H-imidazol-4-yl)ethyl]-3-(1,3-thiazol-2-yl)prop-2-enamide, N-[octadeca-9,12,15-trienoyl]histidine, N-[octadec-9-enoyl]histidine, N-octadecanoylhistidine, 3-(1H-imidazol-4-yl)prop-2-enamide, 3-(1H-imidazol-4-yl)-N-[2-(pyridin-4-yl)ethyl]prop-2-enamide, methyl 2,3-dihydro-1H-indole-2-carboxylate, 3-(1H-imidazol-4-yl)-N-[2-(pyridin-2-yl)ethyl]prop-2-enamide, N-[3,7-dimethylocta-2,6-dien-1-yl]-3-(1H-imidazol-4-yl)prop-2-enamide, N-[2-(1H-imidazol-4-yl)ethyl]-2,3-dihydro-1H-indole-2-carboxamide, 3-(pyrimidin-2-yl)-N-[2-(pyrimidin-2-yl)ethyl]prop-2-enamide, 3,6-bis[(1H-imidazol-4-yl) methyl]piperazine-2,5-dione.

3. A flavor composition comprising a compound as defined in claim 1.

4. The flavour composition according to claim 3, wherein the compound is comprised in between 0.001 and 80 wt % based on the total weight of the composition.

5. A consumer product comprising the compound as defined in claim 1 and a consumer product base.

6. The consumer product according to claim 5, comprising at least 1 ppm of one or more flavor modulating compounds according to formula (I) and/or edible salts thereof.

7. The consumer product according to claim 5 wherein the consumer product is selected from foodstuff and beverages.

8. A compound of formula (I) 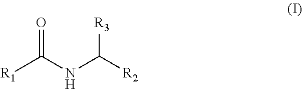
and edible salts thereof, wherein
R₁ is selected from the group consisting of 2-(1H-4-imidazolyl)-ethenyl, 1H-5-indolyl, 2-(1H-5-imidazolyl)-ethenyl, 1-amino-2-(1H-4-imidazolyl)-ethyl, (1,3-thiazol-2-yl)-ethenyl, 2,3-dihydro-1H-indol-2-yl, 2-(pyrimidin-2-yl)ethenyl, heptadecanyl, 1-heptadec-8-enyl, heptadeca-8,11-dienyl, heptadeca-8,11,14-trienyl, 2-(4H-imidazol-2-yl)-ethenyl, 2-(4H-imidazol-2-yl)-ethyl, 2-phenyl-ethenyl, 2-(furan-2-yl)-ethenyl, 2-(thiophen-2-yl)-ethenyl, 2-(thiophen-3-yl)-ethenyl, 2-but-2-enoyl, 2-butyl, 2,6-dimethylhepta-1,5-dienyl, 1,3-benzothiazol-6-yl;
R₂ is selected from the group consisting of (1H-imidazol-4-yl)-methyl, (1H-3-indol-3-yl)-methyl, 4-hydroxybenzyl, methylsulfanylethyl, hydroxymethyl, CH₂—COOH, (pyridin-4-yl)methyl, (pyridin-2-yl) methyl, 1-(2,6-dimethylhepta-1,5-dienyl), 2-(pyrimidin-2-yl)methyl, (1H-imidazol-5-yl)-methyl, (1H-imidazol-2-yl)-methyl, (1H-pyrrol-2-yl)-methyl, phenyl, pyrimidin-5-yl, pyrazin-2-yl;
R₃ is selected from the group consisting of H, COOH, with the proviso that the compound is not 3-(1H-imidazol-4-yl)-N-[2-(1H-imidazol-4-yl)ethyl]prop-2-enamide, histidyltyrosine, histidylhistidine, N-(2-hydroxyethyl)histidinamide, histidyl-β-alanine, N-octadecanoylhistidine, N-[(9Z)-octadec-9-enoyl]histidine.

9. The compound according to claim 8 selected from the group consisting of N-[2-(1H-imidazol-4-yl)ethyl]-1H-indole-5-carboxamide, N-[2-(1H-imidazol-4-yl)ethyl]-1H-indole-5-carboxamide, N-[3-(1H-imidazol-5-yl)prop-2-enoyl]tryptophan, N-[3-(methylsulfanyl)propyl]histidinamide, N-[octadeca-9,12-dienoyl]histidine, N-[2-(1H-imidazol-4-yl)ethyl]-3-(1,3-thiazol-2-yl)prop-2-enamide, N-[octadeca-9,12,15-trienoyl]histidine, 3-(1H-imidazol-4-yl)-N-[2-(pyridin-4-yl)ethyl]prop-2-enamide, 3-(1H-imidazol-4-yl)-N-[2-(pyridin-2-yl)ethyl]prop-2-enamide, N-[3,7-dimethylocta-2,6-dien-1-yl]-3-(1H-imidazol-4-yl)prop-2-enamide, N-[2-(1H-imidazol-4-yl)ethyl]-2,3-dihydro-1H-indole-2-carboxamide, 3-(pyrimidin-2-yl)-N-[2-(pyrimidin-2-yl)ethyl]prop-2-enamide, 3-(4H-imidazol-2-yl)-N-[2-(1H-imidazol-5-yl)ethyl]prop-2-enamide, 3-(1H-imidazol-4-yl)-N-[2-(1H-imidazol-2-yl)ethyl]prop-2-enamide, N-(2-hydroxyethyl)-3-(1H-imidazol-4-yl)propanamide, 3-(1H-imidazol-4-yl)-N-[2-(1H-pyrrol-2-yl)ethyl]prop-2-enamide, N-benzyl-3-(1H-imidazol-4-yl)prop-2-enamide, 3-(1H-imidazol-4-yl)-N-[(pyrimidin-5-yl)methyl]prop-2-enamide, 3-(1H-imidazol-4-yl)-N-[(pyrazin-2-yl) methyl]prop-2-enamide, 3-(furan-2-yl)-N-[2-(1H-imidazol-4-yl)ethyl]prop-2-enamide, N-[2-(1H-imidazol-4-yl)ethyl]-3-(thiophen-2-yl)prop-2-enamide, N-[2-(1H-imidazol-4-yl)ethyl]-3-(thiophen-3-yl)prop-2-enamide, 3-(1H-imidazol-4-yl)-2-{[(2E)-2-methylbut-2-enoyl]amino}propanoic acid, N-[2-(1H-imidazol-4-yl)ethyl]-2-methylbutanamide, N-[2-(1H-imidazol-4-yl)ethyl]-3,7-dimethylocta-2,6-dienamide, 2-{[3-(furan-2-yl)prop-2-enoyl]amino}-3-(1H-imidazol-4-yl)propanoic acid, 3-(1H-imidazol-5-yl)-2-{[3-(1H-imidazol-4-yl) prop-2-enoyl]amino}propanoic acid, N-[2-(1H-imidazol-5-yl)ethyl]-1,3-benzothiazole-6-carboxamide, 3-(1H-imidazol-4-yl)-N-(2-phenylethyl)prop-2-enamide.

10. A method to confer, enhance, improve or modify the flavor properties of a flavor composition or a consumer product, which method comprises adding to said composition or consumer product at least one compound as defined in claim 1 and/or edible salt thereof.

11. A flavor composition comprising a compound as defined in claim 2.

12. A consumer product comprising the compound as defined in claim 2 and a consumer product base.

13. A consumer product comprising the flavor composition as defined in claim 3 and a consumer product base.

14. A consumer product comprising the flavor composition as defined in claim 4 and a consumer product base.

15. The consumer product according to claim 5 comprising at least 20 ppm of one or more flavor modulating compounds according to formula (I) and/or edible salts thereof.

16. The consumer product according to claim 5 comprising at least 50 ppm of one or more flavor modulating compounds according to formula (I) and/or edible salts thereof.

17. The consumer product according to claim 5 comprising at least 70 ppm of one or more flavor modulating compounds according to formula (I) and/or edible salts thereof.

18. The consumer product according to claim 6, wherein the consumer product is selected from foodstuff and beverages.

19. A method to confer, enhance, improve or modify the flavor properties of a flavor composition or a consumer product, which method comprises adding to said composition or consumer product at least one compound as defined in claim 2 and/or edible salt thereof.
